(19) United States
(12) Patent Application Publication
(10) Pub. No.: US 2025/0339467 A1
(43) Pub. Date: Nov. 6, 2025

(54) ITAM DIVERSITY IN CHIMERIC ANTIGEN RECEPTOR POLYPEPTIDES AND METHODS OF USE THEREOF

(71) Applicant: University of Utah Research Foundation, Salt Lake City, UT (US)

(72) Inventors: Matthew Bettini, Salt Lake City, UT (US); Shubhabrata Majumdar, Salt Lake City, UT (US); Hilda Echelibe, Salt Lake City, UT (US)

(21) Appl. No.: 19/198,917

(22) Filed: May 5, 2025

Related U.S. Application Data
(60) Provisional application No. 63/643,350, filed on May 6, 2024.
(60) Provisional application No. 63/733,555, filed on Dec. 13, 2024.

(51) Int. Cl. A61K 35/17; A61K 40/11; A61K 40/24; A61K 40/42; A61P 35/02; C07K 14/705; C07K 14/725; C07K 16/28; C12N 5/0783

(52) U.S. Cl. CPC A61K 35/17; A61K 40/11; A61K 40/24; A61K 40/4211; A61P 35/02; C07K 14/7051; C07K 14/70521; C07K 16/2803; C12N 5/0636; A61K 2239/13; A61K 2239/17; A61K 2239/21; A61K 2239/48; C07K 2317/53; C07K 2317/565; C07K 2317/622; C07K 2319/02; C07K 2319/03

(57) ABSTRACT

Disclosed are CAR polypeptides comprising an antigen binding domain, a transmembrane domain, and an intracellular signaling domain, wherein the intracellular signaling domain comprises a variant CD3 zeta (CD3ζ). Disclosed are nucleic acid sequences capable of encoding any of the disclosed CAR polypeptides. Disclosed are vectors comprising the nucleic acid sequence of the disclosed CAR nucleic acid sequences. Disclosed are cells comprising any of the CAR polypeptides, CAR nucleic acid sequences, or vectors disclosed herein. Disclosed are methods of treating a subject having cancer comprising administering a therapeutically effective amount of a composition comprising a T cell genetically modified to express one or more of the CAR polypeptides disclosed herein to the subject having cancer. Disclosed are methods of using one or more of the disclosed CAR polypeptides.

### CROSS-REFERENCE TO RELATED APPLICATIONS

This application claims the benefit of U.S. Provisional Patent Application Nos. 63/643,350, filed May 6, 2024, and 63/733,555, filed Dec. 13, 2024, each of which is incorporated by reference herein in its entirety.

### STATEMENT REGARDING FEDERALLY SPONSORED RESEARCH

This invention was made with government support under R01 CA281294 awarded by the National Institutes of Health. The government has certain rights in the invention.

### REFERENCE TO SEQUENCE LISTING

The Sequence Listing submitted May 5, 2025 as a text file named “21101.0479U3.xml,” created on Apr. 28, 2025, and having a size of 20,739 bytes is hereby incorporated by reference pursuant to 37 C.F.R. § 1.52 (c) (5).

### BACKGROUND

Chimeric Antigen Receptors (CARs) are by definition an artificial combination of different receptors not naturally optimized for T cell function. The extracellular single-chain variable fragment region (scFV) recognizes tumor protein antigens in an MHC independent manner similar to antibody recognition, while the intracellular region transduces extracellular stimuli into cell fate decisions similar to T Cell Receptor (TCR) signaling. The intracellular domain of the first-generation CAR constructs contained only Signal 1 properties, that is, phosphorylation and signal transduction through the CD3 zeta immunoreceptor tyrosine-based activation motif (ITAM). Second generation CAR constructs included Signal 2 costimulatory molecules such as CD28, 4-1BB and OX40, allowing for full activation and avoidance of anergy 1,2. Whereas the use of CD28 CAR T constructs increased effector function, these CAR T cells had limited persistence and sometimes increased in vivo toxicity. Alternatively, the addition of 4-1BB led to increased CAR T cell persistence in vivo but with slower effector responses. More recently, the optimization of CAR construct design has gained considerable attention.

### BRIEF SUMMARY

Disclosed are CAR polypeptides comprising an antigen binding domain, a transmembrane domain, and an intracellular signaling domain, wherein the intracellular signaling domain comprises a variant CD3 zeta (CD3ζ).

Disclosed are nucleic acid sequences capable of encoding any of the disclosed CAR polypeptides.

Disclosed are vectors comprising the nucleic acid sequence of the disclosed CAR nucleic acid sequences.

Disclosed are cells comprising any of the CAR polypeptides, CAR nucleic acid sequences, or vectors disclosed herein.

Disclosed are methods of treating a subject having cancer comprising administering a therapeutically effective amount of a composition comprising a T cell genetically modified to express one or more of the CAR polypeptides disclosed herein to the subject having cancer.

Disclosed are methods of reducing tumor growth in a subject having cancer comprising administering a therapeutically effective amount of a T cell genetically modified to express one or more of the CAR polypeptides disclosed herein to the subject.

Disclosed are methods of increasing effector function of a T cell comprising genetically modifying the T cell to express one or more of the CAR polypeptides disclosed herein, wherein the T cell has increased effector function.

Disclosed are methods of increasing persistence of a T cell comprising genetically modifying the T cell to express one or more CAR polypeptides disclosed herein, wherein the T cell has increased persistence.

Disclosed are methods of increasing bond lifetime between a T cell and a target antigen comprising genetically modifying the T cell to express one or more of the CAR polypeptides disclosed herein, wherein the T cell and target antigen have an increased bond lifetime.

Disclosed are methods of increasing T cell receptor force in a T cell comprising genetically modifying the T cell to express one or more of the CAR polypeptides disclosed herein, wherein the T cell has an increased TCR force at the CAR.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A-1B shows results of using CD3ζ with all having the same ITAMs. (FIG. 1A) Mice with a single ITAM sequence for all CD3 subunits exhibit major deficiencies in thymic development. (FIG. 1B) Peripheral T cells were activated in vitro with plate-bound CD38 (1 μg/ml each) for 72 h. The incorporation of [3H] was analyzed. Proliferation is graphed as relative to the WT CD3 ITAM transduced control.

FIG. 2A-2B show a schematic of signaling pathway, slip bond kinetics and catch bond kinetics. A) Modified schematic of proposed mechanotransductive signaling pathways in CAR T-Cells. B) Two types of bonds can form between TCR: pMHC, slip bonds and catch bonds.

FIG. 3 shows an example anti-CD19 human CAR design specifying modifications to the CD3 chain ITAMs.

FIGS. 4A-4B show a better survival of z-CCC CAR bearing mice. (FIG. 4A) NRG mice injected with 0.25×10⁶ Nalm6-luc cells were given 2.5×10⁶ CAR T cells 3 days later. Select times of individual mice are shown. (n=9 mice per group supine and prone shown, Average Flux of cage for each group). FIG. 4B shows the probability of survival. FIG. 4C shows all CARs can kill CD19+Nalm6 cells at an E:T ratio of 1:5 in vitro. FIG. 4D shows ITAMs A and C CARs have better anti-tumor efficacy in vitro at a higher tumor burden of 1:20

FIGS. 5A-5B show representative Ca+2 signaling 2PM images of YC3.60 T cells. T cells were placed in a petri-dish with 1% FBS in clear 1×HBSS. Time-lapse image acquired with an Insight XR laser objective 25×/0.95 in water tuned to 840 nm on a Leica SP8 Deep Dive Falcon microscope at 10s intervals. (FIG. 5A, left) Rep. image prior to induced Ca+2 flux. Blue depicts CFP+ cells. (FIG. 5A, right) Rep. image after induced Ca+2 flux w/PMA. Yellow and green cells represent cell fluxes. (FIG. 5B) Distribution of time-integrated intracellular Ca+2 concentrations in T cells n=17, over n=78 frames. Representative of 1 independent experiment.

FIG. 6 shows a schematic of Force measurements.

FIGS. 7A-7B show (FIG. 7A) Force measurements of each ITAM mutant human CAR expressed in Jurkat cells. (FIG. 7B) Peak bond lifetime for each Zeta ITAM mutant CAR after engagement with CD19. *<0.05, **<0.01 Ordinary 1-way ANOVA.

FIG. 8 shows example phosphoproteomics of WT and ITAM mutant Jurkat CAR cells for “TCR sigaling proteins”. Heat map showing the significantly altered (p-value<0.05) phosphopeptides at resting or after 2 min and 30 min stimulation with CD19+Raji cells. The heat map was generated with MetaboAnalyst 5.0 software normalized to an exogenous internal standard phosphopeptide (LIEDAEpYTAK).

FIG. 9 shows ITAM mutant human primary CAR CD4+/CD8+ T cells were cultured with Nalm6 tumor cells and Nalm6 cell counts were determined over a period of 5 days at 1:5 and 1:20 Effector: Target ratio. n=2 separate experiments.

FIG. 10 shows primary human CD8 CAR T cells were stimulated by CD19+Raji cells and blotted for phospho proteins pPLCg1, pZap70, LAT, Erk, and Akt.

FIG. 11 shows wverlap of TCR and CAR signaling cascades.

FIGS. 12A and B shows (FIG. 12A) Peak Bond Lifetime and Force correlates with TCR activation. Left) TCR55b-A50 was mutated to D, E, and used to transduce SKW3 T cells with WT TCR55a. The transfectants were stimulated by KG-1 cells pulsed with titrated HIV peptides for 14 hours. Anti-CD69 staining was performed on the transduced SKW3 T cells and analyzed by flow cytometry. Right) Force curves for TCR55b-A50-D and E were compared to WT. (FIG. 12B) The Calcium flux dynamics in ITAMs A and C CAR primary T cells is higher compared to the WT.

FIG. 13 shows the absence of Zap-70 diminishes bond lifetime with multiple peptides. Force and Bond lifetime was measure with SKW3 T cell line expressing TCR55 in the presence of HIV peptide and Pep20 agonist peptide.

FIG. 14A-14C show the absence of Zap-70 diminishes bond lifetime of CARs. FIG. 14A) Force and Bond lifetime was measured with CAR ITAM Zeta CCC and with CCC ZAP70 KO Jurkat cells. FIG. 14B) Comparison of Force and Bond lifetime with CAR Zeta CCC and XXC Jurkat cells. FIG. 14C) Comparison of Force and Bond lifetime with Zeta AAA and AXX Jurkat cells.

FIGS. 15A-15C show ITAM mutant CAR ex vivo effector function. At Day-3, 1×10⁶ E□-ALL01 cells were injected i.v. into RAG−/−mice. Day 0, 1×10⁶ CD4+/CD8+CAR T cells were injected i.v. (FIG. 15A) At Day 7, CAR T cells were isolated from the spleen and stimulated with PMA/Ion and stained by ICS for CD8, TNFa and IFNg and (FIG. 15B) IL-2 expression. (FIG. 15C) At day 14, CAR T cells were isolated from the bone marrow and stained with CD8 anti-PD1 and anti-Tim3. *p<0.05 Dunnett's multiple comparisons test.

FIG. 16 shows tumor burden of Z-CCC compared to Z-AXX and Z-XXC CAR treated mice. NRG mice injected with 0.25×10⁶ Nalm6-luc cells were given 2.5×10⁶ Single and Triple zeta ITAM CAR T cells 3 days later. Select times of individual mice are shown.

FIG. 17 shows ITAMs A & C CAR T cells have a higher number of CAR infiltrating lymphocytes following stimulation in vivo. Data from n=2, 6 mice per group; One-way ANOVA with Dunnett's correction test. All comparisons are done relative to z-WT.

FIG. 18 shows ITAM-C CAR T cells express a higher frequency of Ki67 in vivo indicating enhanced proliferation.

FIG. 19 shows ITAMs A & C CD4+CAR T cells have a higher frequency of IL7R+ cells suggesting better survival and generation of memory T cells.

FIG. 20 shows ITAMs A & C CAR T cells have lower expression of markers associated with terminal differentiation, suggesting increased functionality.

FIGS. 21A and 21B show a construct design for diversity restricted ITAM CARs and their expression in primary CD8 T-cells. FIG. 21A) Schematic representation of the construct design for the ITAM restricted CARs along with the individual amino acid sequences of the individual ITAMs. FIG. 21B) Flow plots showing CAR expression in positive correlation with reporter Thy 1.1 expression. CAR expression detected using an antibody targeting the conserved G4S sequence.

FIGS. 22A-22F show ITAM restricted CARs exhibit differential signaling. FIG. 22A) Representative immunoblots of CAR proximal signaling kinetics from whole lysate of CD8+CAR-T cells stimulated with CD19+ dynabeads. FIG. 22B-D) Relative quantification of proximal signaling molecules at peak signaling time-point of 2.5 min. FIG. 22E) Representative ratiometric Ca2+ response curve in Indo-1 labelled CD8+CAR T-cells stimulated with CD19+E-u cells and its relative quantification of median Ca2+ response in CD8+CAR-T cells as area under the curve. FIG. 22F) Nur77 expression quantified in CD8+CAR-T cells stimulated with CD19+E-u cells for 6 hrs at 1:2 E:T ratio. Each dot represents an individual mouse donor. Normalized data was analyzed using one-sample T-test. Red asterisk designates 2.5 min timepoint for b-c. Significance representation *p<0.05, **p<0.01.

FIGS. 23A-23E show in vitro functional characterization of ITAM restricted CAR-T cells suggest Zeta B and Zeta C CARs have weaker functional activation profile upon antigen encounter. FIG. 23A) Representative Specific lysis of CD19+E-u tumor cells in a 24-hour dye release co-culture assay. n=3. FIG. 23B) CAR-T cell survival 24 hours post-killing in 1:2 E: T ratio co-culture with CD19+E-u tumor cells. Data pooled from 2 independent experiments.

FIG. 23C) CD107a degranulation assay with CD8+CAR-T cells co-cultured with CD19+E-u cells at a 1:2 E:T ratio for 4 hrs. Representative (left) and normalized to WT group (right). Data pooled from 4 independent experiments. FIG. 23D-E) Cytokine activation profile of CD8+CAR-T cells stimulated with CD19+E-u cells in 1:2 E:T ratio for 6 hrs in presence of BFA and monensin. Data pooled from 3 independent experiments. Each dot is representative of an individual mouse donor. Normalized data analyzed using one sample T-test. Cytokine expression percentages analyzed using one-way ANOVA followed by a post-hoc Dunnett's test. Significance representation *p<0.05, **p<0.01.

FIGS. 24A-24D shows long-term in vitro stimulation of ITAM restricted CARs suggest Zeta B are more prone to exhaustion and Zeta C CARs are less prone to exhaustion. FIG. 24A) Experimental setup for in vitro exhaustion assay. CD8+CAR-T cells were stimulated with CD19 beads in the presence of IL-2 for 7 days. Stimulations were repeated every 3 days and cells were analyzed on Day 7. FIG. 24B-C) Representative flow plots and combined data of CD8+CAR T-cells expressing inhibitory receptors. FIG. 24D) Relative expression levels of TCF1 and IL7R in CD8+CAR-T cells at Day7. Data pooled from two independent experiments. Each dot represents an individual mouse donor. Normalized data analyzed using one sample T-test. Percentages were analyzed using one-way ANOVA with post-hoc Dunnett's test. Significance representation *p<0.05.

FIGS. 25A-25C show ITAM restricted CARs exhibit differential signaling. Fold change time course of CAR proximal signaling kinetics from whole lysate of CD8+CAR-T cells stimulated with CD19+dynabeads. A) pZap70/total Zap70; B) pLAT/total LAT; C) pPLCγ/total PLCγ.

FIG. 26 shows in vitro functional characterization of ITAM restricted CAR-T cells suggest Zeta B and Zeta C CARs have weaker functional activation profile upon antigen encounter. IL-2 Cytokine profile of CD8+CAR-T cells stimulated with CD19+E-u cells in 1:2 E: T ratio for 6 hrs in presence of BFA and monensin. Data pooled from 3 independent experiments.

FIGS. 27A-C shows long-term in vitro stimulation of ITAM restricted CARs suggest Zeta B are more prone to exhaustion and Zeta C CARs are less prone to exhaustion. Normalized expression of PD1 (FIG. 27A), Tim3 (FIG. 27B) and Lag3 (FIG. 27C) in CD8+CAR-T cells at Day7 compared to WT. Data pooled from two independent experiments. Each dot represents an individual mouse donor.

FIGS. 28A and 28B show ITAM C CAR T cells are more persistent and efficacious following repeated tumor challenges. FIG. 28A) schematic of experimental design; FIG. 28B) in vivo study results.

FIGS. 29A and 29B show CD19 specific CAR ITAM combinations AAC, CCA and BBC kill Nalm6 B cell tumors in vitro. FIG. 29A is a 1:10 dilution and FIG. 29B is a 1:20. CCA kills better than AAB, AAC,BBC and very similar to CCC which is the best CAR construct in vivo thus far. This data shows that the 27 unique combinations of the ITAMs can be pursued and is distinct from the AAA, BBB, CCC of the original constructs.

FIG. 30 shows an experiment optimizing HER2-Specific CAR T cell ITAM sequences to increase solid tumor clearance.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an antigen binding domain” includes a plurality of such antigen binding domains, reference to “the antigen binding domain” is a reference to one or more antigen binding domains and equivalents thereof known to those skilled in the art, and so forth.

The word “or” as used herein means any one member of a particular list and also includes any combination of members of that list.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” acute lymphoblastic leukemia may refer to inhibiting survival, growth, and/or spread of the cancer cells. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, the terms “administering” and “administration” refer to any method of providing a disclosed virus or composition of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

The terms “variant” and “mutant” are used interchangeably herein. As used herein, the term “variant” refers to a modified nucleic acid or protein which displays the same characteristics when compared to a reference nucleic acid or protein sequence. A variant can be at least 65, 70, 75, 80, 85, 90, 95, or 99 percent homologous to a reference sequence. In some aspects, a reference sequence can be a fragment of one or more of the disclosed sequences. A “variant” can mean a difference in some way from the reference sequence other than just a simple deletion of an N- and/or C-terminal amino acid. A variant can also be a difference in the nucleotide sequence. Variants can also or alternatively include at least one substitution and/or at least one addition; there may also be at least one deletion. Alternatively or in addition, variants can comprise modifications, such as non-natural residues at one or more positions with respect to a reference nucleic acid or protein.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Chimeric Antigen Receptor (CAR) Polypeptide

Disclosed are CAR polypeptides comprising an antigen binding domain, a transmembrane domain, and an intracellular signaling domain, wherein the intracellular signaling domain comprises a variant CD3 zeta (CD3ζ). In some aspects, the entire CAR polypeptide is composed of human proteins. In some aspects, one or more of the antigen binding domain, transmembrane domain, or intracellular signaling domain are human. In some aspects, the intracellular signaling domain is derived from human proteins. In some aspects, a variant CD3ζ can be any CD3 that differs from human wild type CD3ζ. In some aspects, a variant CD3ζ can be any CD3ζ that is missing one or more of ITAMS CD3ζa, CD3ζb, or CD3 ζc.

### 1. Antigen Binding Domain

In some aspects, the antigen binding domain is an antibody fragment or an antigen-binding fragment that specifically binds to a target antigen. In some instances, the antigen binding domain can be any recombinant or engineered protein domain capable of binding the target antigen.

In some instances, the antigen binding domain can be a Fab or a single-chain variable fragment (scFv) of an antibody that specifically binds to a target antigen. In some instances, the scFv, comprising both the heavy chain variable region and the light chain variable region, can comprise the N-terminal region of the heavy chain variable region linked to the C-terminal region of the light chain variable region. In some instances, the scFv comprises the C-terminal region of the heavy chain variable region linked to the N-terminal region of the light chain variable region.

In some aspects, the target antigen can be a tumor-associated antigen. In some aspects, the target antigen can be, but is not limited to, CD19, CD229, BCMA, ROR1, and EGFRVIII, CD38, CD123, HER2, carbonic anhydrase IX, MS4A1, CD22, TNFRSF17, SLAMF7, TNFRSF8, CD33, CLEC12A, GPC3, B4GALNT1, ERBB2, EGFR, CD34, FAP, ROR2, AXL, IL3RA, KIT, PROM1, KDR, EPHA2, CD274, MET, MME, FOLH1, GPC3, TNFRSF10B, SDC1, MUC1, EPHA2, EGFR, TNFRSF10B, CD4, MUC16, PSCA, CLDN18, FOLR1, MSLN, MET, EPCAM, or CEACAM5.

In some aspects, the target antigen is CD19, thus the antigen binding domain is a CD19 antigen binding domain.

In some aspects, the CD19 antigen binding domain comprises the amino acid sequence of DIQMTQTTSSLSASLGDRVTISCRASQDISKYLNWYQQKPDGTVKLLIYHTSRLHSGVPS RFSGSGSGTDYSLTISNLEQEDIATYFCQQGNTLPYTFGGGTKLELKRGGGGSGGGGSG GGGSGGGGSEVQLQQSGPGLVAPSQSLSVTCTVSGVSLPDYGVSWIRQPPRKGLEWLG VIWGSETTYYNSALKSRLTIIKDNSKSQVFLKMNSLQTDDTAIYYCAKHYYYGGSYAM DY (SEQ ID NO:4). The first sequence in regular font is the variable heavy chain; the underlined sequence is the linker; the bold sequence is the variable light chain.

In some aspects, the CD19 antigen binding domain comprises a heavy chain immunoglobulin variable region comprising a complementarity determining region 1 (CDR1) comprising the sequence of GVSLPDYGVS (SEQ ID NO:5); a CDR2 comprising the sequence of VIWGSETTYYNSALKS (SEQ ID NO:6); and a CDR3 comprising the sequence of KHYYYGGSYAMDY (SEQ ID NO:7).

In some aspects, the CD19 antigen binding domain comprises a light chain immunoglobulin variable region comprising a CDR1 comprising the sequence of RASQDISKYLN (SEQ ID NO:8); a CDR2 comprising the sequence of HTSRLHS (SEQ ID NO: 9); and a CDR3 comprising the sequence of QQGNTLPYT (SEQ ID NO:10).

In some aspects, the CD19 antigen binding domain is a single-chain variable fragment (scFv) of an antibody that specifically binds CD19, wherein the CDR1 sequence of the V_{H} domain comprises the amino acid sequence SEQ ID NO:5; the CDR2 sequence of the V_{H} domain comprises the amino acid sequence SEQ ID NO:6; the CDR3 sequence of the V_{H} domain comprises the amino acid sequence SEQ ID NO:7; the CDR1 sequence of the V_{L} comprises the amino acid sequence SEQ ID NO:8; the CDR2 sequence of the V_{L} domain comprises the amino acid sequence SEQ ID NO:9; and the CDR3 sequence of the V_{L} domain comprises the amino acid sequence SEQ ID NO:10.

In some aspects, the target antigen is neuroblastoma GD2, thus the antigen binding domain is a neuroblastoma GD2 antigen binding domain.

In some aspects, the neuroblastoma GD2 antigen binding domain comprises the amino acid sequence of

(SEQ ID NO: 21)
MEFGLSWLFLVAILKGVQCSRDILLTQTPLSLPVSLGDQASISCRSSQSL
VHRNGNTYLHWYLQKPGQSPKLLIHKVSNRFSGVPDRFSGSGSGTDFTLK
ISRVEAEDLGVYFCSQSTHVPPLTFGAGTKLELKRADAAPTVSIFPGSGG
GGSGGEVKLQQSGPSLVEPGASVMISCKASGSSFTGYNMNWVRQNIGKSL
EWIGAIDPYYGGTSYNQKFKGRATLTVDKSSSTAYMHLKSLTSEDSAVYY
CVSGMEYWGQGTSVTVSSAKTTPPSVYGRVTVSSA.

### 2. Transmembrane Domain

In some instances, the transmembrane domain comprises an immunoglobulin Fc domain. In some instances, the immunoglobulin Fc domain can be an immunoglobulin G Fc domain.

In some aspects, the transmembrane domain comprises a transmembrane domain of a protein chosen from the alpha, beta, or zeta chain of T-cell receptor, CD28, OX40, H2-Kb, CD3 epsilon, CD45, CD4, CD5, CD7, CD8, CD9, CD16, CD22, CD33, CD37, CD64, CD80, CD86, CD134, CD137, CD154, or immunoglobulin Fc domain. In some instances, the transmembrane domain comprises a CD8a domain, CD3ζ, FcεR1γ, CD4, CD7, CD28, OX40, or H2-Kb.

In some instances, the transmembrane domain can be located between the antigen binding domain and the intracellular signaling domain.

### 3. Intracellular Signaling Domain

In some aspects, the intracellular signaling domain is a T cell signaling domain, specifically a CD3 signaling domain. In some instances, the CD3ζ signaling domain is the intracellular domain of CD33. In some instances, CD3ζ signaling domain is a variant CD3 signaling domain.

In some aspects, the CD3ζ signaling domain comprises three immunoreceptor tyrosine activation motifs (ITAMs), A, B, and C, also referred to as CD3ζa, CD3ζb, and CD3 ζc. In some instances, a variant CD3ζ signaling domain comprises a different variation of the A, B, and C ITAMs compared to a wild type CD3ζ signaling domain.

In some aspects, the variant CD3ζ comprises two or more of ITAMs CD3ζa, CD3ζb, or CD3 ζc. In some aspects, the variant CD3ζ comprises two or more of ITAMs CD3ζa. In some aspects, the variant CD3 comprises two CD3ζa ITAMs and one CD3ζb ITAM, thus, the variant CD3ζ comprises CD3ζa, CD3ζa, CD3ζb. In some aspects, the variant CD3ζ comprises two CD3ζa ITAMs and one CD3 ζc ITAM, thus, the variant CD3ζ comprises CD3ζa, CD3ζa, CD3 ζc.

In some aspects, the variant CD3 comprises two or more of ITAMs CD3ζb. In some aspects, the variant CD3ζ comprises two CD3ζb ITAMs and one CD3ζa ITAM, thus the variant CD3ζ comprises CD3ζb, CD3ζb, CD3ζa. In some aspects, the variant CD3ζ comprises two CD3ζb ITAMs and one CD3 ζc ITAM, thus, the variant CD3ζ comprises CD3ζb, CD3ζb, CD3 ζc.

In some aspects, the variant CD3ζ comprises two or more of ITAMs CD3ζc. In some aspects, the variant CD3ζcomprises two CD3ζc ITAMs and one CD3ζa ITAM, thus, the variant CD3ζ comprises CD3ζc, CD3ζc, CD3ζa. In some aspects, the variant CD3ζ comprises two CD3 ζc ITAMs and one CD3ζb ITAM, thus, the variant CD3ζ comprises CD3ζc, CD3ζc, CD3ζb.

In some aspects, the variant CD3ζ does not comprise one or more of ITAMs CD3ζa, CD3ζb, and CD3 ζc. For example, the variant CD3ζ is not CD3ζa, CD3ζb, CD3ζc. In other words, the variant CD3ζ can comprise one or two, but not all three, of ITAMs CD3ζa, CD3ζb, and CD3 ζc. In some aspects, the variant CD3ζ comprises three ITAMs but they are not CD3ζa, CD3ζb, and CD3 ζc. In some aspects, the variant CD3ζ comprises three CD3c ITAMs. In some aspects, the variant CD3ζ comprises three CD3ζb ITAMs. In some aspects, the variant CD3ζ comprises three CD3ζa ITAMs. In some aspects, the variant CD3ζ comprises only three ITAMS, for example, three CD3ζc ITAMs, three CD3ζb ITAMs, or three CD3ζa ITAMs.

In some aspects, the one or more CD3ζa ITAMs comprise the amino acid sequence of QLYNELNLGRREEYDVL (SEQ ID NO:1), or a variant thereof. In some aspects, the one or more CD3ζb ITAMs comprise the amino acid sequence of GLYNELQKDKMAEAYSEI (SEQ ID NO: 2), or a variant thereof. In some aspects, the one or more CD3ζc ITAMs comprise the amino acid sequence of GLYQGLSTATKDTYDAL (SEQ ID NO:3), or a variant thereof. In some aspects, a variant thereof can be a CD3ζa, CD3ζb, and/or CD3 ζc having at least 70, 75, 80, 85, 90, 95, or 99% identity to SEQ ID NO: 1, 2, and/or 3.

In some instances, the intracellular signaling domain comprises a co-stimulatory signaling region. In some instances, the co-stimulatory signaling region can comprise the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some instances, the intracellular signaling domain comprises a variant CD3ζ signaling domain and a co-stimulatory signaling region, wherein the co-stimulatory signaling region comprises the cytoplasmic domain of CD28, 4-1BB, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

### 4. Hinge Region

Any of the disclosed CAR polypeptides can further comprise a hinge region. For example, disclosed are CAR polypeptides comprising an antigen binding domain, a transmembrane domain, and an intracellular signaling domain and further comprising a hinge region.

In some instances, the hinge region can be located between the antigen binding domain and the transmembrane domain.

In some instances, the hinge region allows for the antigen binding domain to bind to the antigen. For example, the hinge region can increase the distance of the binding domain to the cell surface and provide flexibility.

In some aspects, the hinge region is from CD3zeta, CD4, CD8, CD28, or heavy chain of immunoglobulin.

### 5. Tag

In some instances, any of the disclosed CAR polypeptides can further comprise a tag.

In some instances, the tag can be located between the antigen binding domain and the transmembrane domain or between the antigen binding domain and a hinge region. In some instances, the tag can be a hemagglutinin tag, histidine tag, glutathione-S-transferase tag, or fluorescent tag. For example, the tag can be any sequence/molecule/compound capable of aiding in the purification of the CAR polypeptide or capable of detecting the CAR polypeptide.

### C. Nucleic Acid Sequences

Disclosed are nucleic acid sequences capable of encoding any of the disclosed CAR polypeptides. For example, disclosed are nucleic acid sequences capable of encoding a CAR polypeptide comprising a antigen binding domain, a transmembrane domain, and an intracellular signaling domain.

In some aspects, the disclosed nucleic acid sequences can be DNA or RNA sequences.

### 1. Antigen Binding Domain

Disclosed are nucleic acid sequence that encode any of the antigen binding domains described herein.

In some aspects, the CD19 antigen binding domain comprises the amino acid sequence of

GACATCCAGATGACACAGACTACATCCTCCCTGTCTGCCTCTCTGGGAGA
CAGAGTCACCATCAGTTGCAGGGCAAGTCAGGACATTAGTAAATATTTAA
ATTGGTATCAGCAaAAACCAGATGGAACTGTTAAACTCCTGATCTACCAT
ACATCAAGATTACACTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGGTAATACGCTTCCGTACACGTTCGGAGGG
GGGACCAAGCTGGAGCTGAAACGTGGTGGTGGTGGTTCTGGTGGTGGTGG
TTCTGGCGGCGGCGGCTCCGGTGGTGGTGGATCCGAGGTGCAGCTGCAGC
AGTCTGGACCTGGCCTGGTGGCGCCCTCACAGAGCCTGTCCGTCACATGC
ACTGTCTCAGGGGTCTCATTACCCGACTATGGTGTAAGCTGGATTCGCCA
GCCTCCACGAAAGGGTCTGGAGTGGCTGGGAGTAATATGGGGTAGTGAAA
CCACATACTATAATTCAGCTCTCAAATCCAGACTGACCATCATCAAGGAC
AACTCCAAGAGCCAAGTTTTCTTAAAAATGAACAGTCTGCAAACTGATGA
CACAGCCATTTACTACTGTGCCAAACATTATTACTACGGTGGTAGCTATG
CTATGGACTAC

In some aspects, the CD19 antigen binding domain comprises a heavy chain immunoglobulin variable region comprising a complementarity determining region 1 (CDR1) comprising the sequence of GGGGTCTCATTACCCGACTATGGTGTAAGC (SEQ ID NO:11; a CDR2 comprising the sequence of GTAATATGGGGTAGTGAAACCACATACTATAATTCAGCTCTCAAATCC (SEQ ID NO: 12); and a CDR3 comprising the sequence of AAACATTATTACTACGGTGGTAGCTATGCTATGGACTAC (SEQ ID NO:13).

In some aspects, the CD19 antigen binding domain comprises a light chain immunoglobulin variable region comprising a CDR1 comprising the sequence of AGGGCAAGTCAGGACATTAGTAAATATTTAAAT (SEQ ID NO: 14); a CDR2 comprising the sequence of CATACATCAAGATTACACTCA (SEQ ID NO:15); and a CDR3 comprising the sequence of CAACAGGGTAATACGCTTCCGTACACG (SEQ ID NO:16).

In some aspects, the CD19 antigen binding domain is a single-chain variable fragment (scFv) of an antibody that specifically binds CD19, wherein the CDR1 sequence of the V_{H} domain comprises the nucleic acid sequence SEQ ID NO: 11; the CDR2 sequence of the V_{H} domain comprises the nucleic acid sequence SEQ ID NO:12; the CDR3 sequence of the V_{H} domain comprises the nucleic acid sequence SEQ ID NO: 13; the CDR1 sequence of the V_{L} comprises the nucleic acid sequence SEQ ID NO: 14; the CDR2 sequence of the V_{L} domain comprises the nucleic acid sequence SEQ ID NO: 15; and the CDR3 sequence of the V_{L} domain comprises the nucleic acid sequence SEQ ID NO:16.

In some aspects, antigen binding domain is a neuroblastoma GD2 antigen binding domain. In some aspects, the neuroblastoma GD2 antigen binding domain comprises the amino acid sequence of

(SEQ ID NO: 17)
ATGGAGTTTGGGCTGAGCTGGCTTTTTCTTGTGGCTATTTTAAAAGGTGT
CCAGTGCTCTAGAGATATTTTGCTGACCCAAACTCCACTCTCCCTGCCTG
TCAGTCTTGGAGATCAAGCCTCCATCTCTTGCAGATCTAGTCAGAGTCTT
GTACACCGTAATGGAAACACCTATTTACATTGGTACCTGCAGAAGCCAGG
CCAGTCTCCAAAGCTCCTGATTCACAAAGTTTCCAACCGATTTTCTGGGG
TCCCAGACAGGTTCAGTGGCAGTGGATCAGGGACAGATTTCACACTCAAG
ATCAGCAGAGTGGAGGCTGAGGATCTGGGAGTTTATTTCTGTTCTCAAAG
TACACATGTTCCTCCGCTCACGTTCGGTGCTGGGACCAAGCTGGAGCTGA
AACGGGCTGATGCTGCACCAACTGTATCCATCTTCCCAGGCTCGGGCGGT
GGTGGGTCGGGTGGCGAGGTGAAGCTTCAGCAGTCTGGACCTAGCCTGGT
GGAGCCTGGCGCTTCAGTGATGATATCCTGCAAGGCTTCTGGTTCCTCAT
TCACTGGCTACAACATGAACTGGGTGAGGCAGAACATTGGAAAGAGCCTT
GAATGGATTGGAGCTATTGATCCTTACTATGGTGGAACTAGCTACAACCA
GAAGTTCAAGGGCAGGGCCACATTGACTGTAGACAAATCGTCCAGCACAG
CCTACATGCACCTCAAGAGCCTGACATCTGAGGACTCTGCAGTCTATTAC
TGTGTAAGCGGAATGGAGTACTGGGGTCAAGGAACCTCAGTCACCGTCTC
CTCAGCCAAAACGACACCCCCATCAGTCTATGGAAGGGTCACCGTCTCTT
CAGCG.

### 2. Transmembrane Domain

In some instances, the transmembrane domain comprises a nucleic acid sequence that encodes an immunoglobulin Fc domain. In some instances, the immunoglobulin Fc domain can be an immunoglobulin G Fc domain.

In some instances, the transmembrane domain comprises a nucleic acid sequence that encodes a protein chosen from the alpha, beta, or zeta chain of T-cell receptor, CD28, OX40, H2-Kb, CD3 epsilon, CD45, CD4, CD5, CD7, CD8, CD9, CD16, CD22, CD33, CD37, CD64, CD80, CD86, CD134, CD137, CD154, or immunoglobulin Fc domain.

In some instances, the transmembrane domain comprises a nucleic acid sequence that encodes a CD8a domain, CD3ζ, FcεR1γ, CD4, CD7, CD28, OX40, or H2-Kb.

In some instances, the nucleic acid sequence that encodes the transmembrane domain can be located between the nucleic acid sequence that encodes the TM4SF1 antigen binding domain and the nucleic acid sequence that encodes the intracellular signaling domain.

### 3. Intracellular Domain

In some instances, the intracellular signaling domain can be a nucleic acid sequence encoding a T cell signaling domain. For example, the intracellular signaling domain can comprise a nucleic acid sequence that encodes a CD3ζ signaling domain. In some instances, the CD3ζ signaling domain is the intracellular domain of CD3ζ. In some instances, CD3ζ signaling domain is a variant CD3ζ signaling domain.

In some aspects, the nucleic acid sequence encodes a CD3ζ signaling domain comprising CD3ζa, CD3ζb, and CD3 ζc. In some instances, the nucleic acid sequence encodes a variant CD3ζ signaling domain comprising a different variation of the A, B, and C ITAMs compared to a wild type CD3ζ signaling domain.

In some aspects, the nucleic acid sequence encodes a variant CD3 comprising two or more of ITAMs CD3ζa, CD3ζb, or CD3ζc. In some aspects, the nucleic acid sequence encodes a variant CD3ζ comprising two or more of ITAMs CD3ζa. In some aspects, the nucleic acid sequence encodes a variant CD3ζ comprising two CD3ζa ITAMs and one CD3ζb ITAM, thus, the variant CD3 comprises CD3ζa, CD3ζa, CD3ζb. In some aspects, the nucleic acid sequence encodes a variant CD3ζ comprising two CD3ζa ITAMs and one CD3ζc ITAM, thus, the variant CD3ζ comprises CD3ζa, CD3ζa, CD3ζc.

In some aspects, the nucleic acid sequence encodes a variant CD3ζ comprising two or more of ITAMs CD3ζb. In some aspects, the nucleic acid sequence encodes a variant CD3ζ comprising two CD3ζb ITAMs and one CD3ζa ITAM, thus the variant CD3ζ comprises CD3ζb, CD3ζb, CD3ζa. In some aspects, the nucleic acid sequence encodes a variant CD3ζ comprising two CD3ζb ITAMs and one CD3ζc ITAM, thus, the variant CD3 comprises CD3ζb, CD3ζb, CD3 ζc.

In some aspects, the nucleic acid sequence encodes a variant CD34 comprising two or more of ITAMs CD3c. In some aspects, the nucleic acid sequence encodes a variant CD33 comprising two CD3ζc ITAMs and one CD3ζa ITAM, thus, the variant CD3 comprises CD3ζc, CD3ζc, CD3ζa. In some aspects, the nucleic acid sequence encodes a variant CD3ζ comprising two CD3ζc ITAMs and one CD3ζb ITAM, thus, the variant CD3ζ comprises CD3ζc, CD3ζc, CD3ζb.

In some aspects, the nucleic acid sequence encodes a variant CD3ζ that does not comprise one or more of ITAMs CD3ζa, CD3ζb, and CD3 ζc. For example, the variant CD3ζ is not CD3ζa, CD3ζb, CD3 ζc. In other words, the nucleic acid sequence encodes a variant CD3ζ comprising one or two, but not all three, of ITAMs CD3ζa, CD3ζb, and CD3c. In some aspects, the nucleic acid sequence encodes a variant CD3ζcomprising three ITAMs but they are not one each of CD3ζa, CD3ζb, and CD3ζc. In some aspects, the nucleic acid sequence encodes a variant CD3ζ comprises three CD3ζc ITAMs. In some aspects, the nucleic acid sequence encodes a variant CD3ζ comprising three CD3ζb ITAMs. In some aspects, the nucleic acid sequence encodes a variant CD3ζ comprising three CD3ζa ITAMs. In some aspects, the nucleic acid sequence encodes a variant CD3ζ comprising only three ITAMS, for example, three CD3ζc ITAMs, three CD3ζb ITAMs, or three CD3ζa ITAMs.

In some aspects, the one or more CD3ζa ITAMs comprise the nucleic acid sequence of CAACTTTATAATGAACTTAACCTCGGTCGTCGAGAAGAATACGATGTTCTA (SEQ ID NO: 18), or a variant thereof. In some aspects, the one or more CD3ζb ITAMs comprise the nucleic acid sequence of GGCCTGTACAATGAACTGCAGAAAGATAAGATGGCGGAGGCCTACAGTGAGATT (SEQ ID NO:19), or a variant thereof. In some aspects, the one or more CD3ζc ITAMs comprise the nucleic acid sequence of GGCCTTTACCAGGGTCTCAGTACAGCCACCAAGGACACCTACGACGCCCTT (SEQ ID NO: 20), or a variant thereof. In some aspects, a variant thereof can be a CD3ζa, CD3ζb, and/or CD3 ζc having at least 70, 75, 80, 85, 90, 95, or 99% identity to SEQ ID NO: 18, 19, 20.

In some instances, the intracellular signaling domain comprises a variant CD3ζ signaling domain and a co-stimulatory signaling region, wherein the co-stimulatory signaling region comprises the cytoplasmic domain of CD28, 4-1BB, CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

### 4. Hinge Region

In some instances, the hinge region can be a nucleic acid sequence encoding a hinge region. For example, disclosed are nucleic acid sequences that encode the hinge region portion of CD3zeta, CD4, CD8, CD28, or heavy chain of immunoglobulin.

In some instances, the nucleic acid sequence that encodes the hinge region can be located between the nucleic acid sequence that encodes the TM4SF1 antigen binding domain and the nucleic acid sequence that encodes the transmembrane domain.

### D. Vectors

Disclosed are vectors comprising the nucleic acid sequence of the disclosed CAR nucleic acid sequences. Disclosed are vectors comprising a nucleic acid sequence the encodes any of the disclosed CAR polypeptides. In some aspects, the vector can be selected from the group consisting of a DNA, a RNA, a plasmid, and a viral vector. In some instances, the vector can comprise a promoter.

In some aspects, the vector can be an expression vector. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

In some aspects, the vector can be a viral vector. For example, the viral vector can be a lentiviral vector. In some aspects, the vector can be a non-viral vector, such as a DNA based vector.

i. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding one or more of the disclosed CAR polypeptides.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORTI plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)). The three examples employ bacterial genes under eukaryotic control to convey resistance to the appropriate drug G418 or neomycin (geneticin), xgpt (mycophenolic acid) or hygromycin, respectively. Others include the ncomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### E. Cells

Disclosed are cells comprising any of the CAR polypeptides, CAR nucleic acid sequences, or vectors disclosed herein. These cells can be considered genetically modified. In some aspects, the cells can be a cell line.

In some instances, the cell can be a T cell. For example, the T cell can be a CD8+ T cell. In some instances, the cell can be a mammalian cell, such as a human cell.

Thus, disclosed are T cells expressing one of the CAR polypeptides disclosed herein.

In some aspects, the cells can be eukaryotic or prokaryotic. In some aspects, the cells are mammalian cells. In some aspects, the cell is a human cell. In some aspects, the cell is a αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof. In some aspects, the regulatory T cell is a CD8+ T cell or CD4+ T cell.

Disclosed are T cells expressing the one or more of the CAR polypeptides disclosed herein.

### F. Compositions

Disclosed are compositions comprising the disclosed CAR polypeptides, nucleic acid sequences, vectors, or cells. Disclosed are compositions comprising a CAR polypeptide comprising an antigen binding domain, a transmembrane domain, and an intracellular signaling domain. Disclosed are compositions comprising a nucleic acid construct encoding a CAR polypeptide comprising an antigen binding domain, a transmembrane domain, and an intracellular signaling domain. Also disclosed are compositions comprising a vector comprising a nucleic acid construct, wherein the nucleic acid construct comprises a nucleic acid sequence encoding a CAR polypeptide comprising an antigen binding domain, a transmembrane domain, and an intracellular signaling domain.

The disclosed compositions can further comprise a pharmaceutically acceptable carrier.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed CAR polypeptides, nucleic acid sequences, cells or vectors can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed peptides described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed peptides. That is, a pharmaceutical composition can be provided comprising a therapeutically effective amount of at least one disclosed peptide or at least one product of a disclosed method and a pharmaceutically acceptable carrier.

In certain aspects, the disclosed pharmaceutical compositions comprise the disclosed peptides (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The peptides described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the case in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their case of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed peptide (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### G. Methods of Treating

Disclosed are methods of treating a subject having cancer comprising administering a therapeutically effective amount of a composition comprising a T cell genetically modified to express one or more of the CAR polypeptides disclosed herein to the subject having cancer.

In some aspects, the cancer is acute lymphoblastic leukemia. In some aspects, the CAR polypeptide comprises a CD19, CD20, CD22, CD24 or CD79a antigen binding domain. In some aspects, each of these antigens can be a biomarker for acute lymphoblastic leukemia.

In some aspects, the methods of treating comprise the killing of a tumor cell in the subject having cancer. In some aspects, a tumor cell is targeted by a T cell genetically modified to express a CAR polypeptides comprising an antigen binding domain, a transmembrane domain, and an intracellular signaling domain, wherein the intracellular signaling domain comprises a variant CD3 zeta (CD3ζ).

In some aspects, the methods result in clearance of tumor cells in the subject having cancer. In some aspects, the methods result in a reduction of tumor growth in the subject having cancer. In some aspects, the methods result in an increase in effector function of the genetically modified T cell, thus providing better response to cancer cells.

In some aspects, T cells comprising one or more of the disclosed CAR polypeptides can treat a subject having cancer better than a T cell comprising a CAR polypeptide with wild type ITAMs. In some aspects, T cells comprising one or more of the disclosed CAR polypeptides can treat a subject having cancer better than a T cell comprising a CAR polypeptide with a single ITAM instead of three ITAMs.

In some aspects, the subject having cancer can have, but is not limited to, acute lymphoblastic leukemia, B cell lymphoma, multiple myeloma, neuroblastoma, glioblastoma, gastric cancer, prostate carcinoma, renal carcinoma, colorectal cancer, hepatocellular carcinoma, non-small cell lung cancer, bladder cancer, breast cancer, mammary cancer, melanoma, squamous cell carcinoma, head and neck squamous cell carcinomas, pancreatic cancer, sarcoma, ovarian cancer, or mantle cell lymphoma.

### H. Methods of Reducing Tumor Growth

Disclosed are methods of reducing tumor growth in a subject having cancer comprising administering a therapeutically effective amount of a T cell genetically modified to express one or more of the CAR polypeptides disclosed herein to the subject.

Disclosed are methods of reducing tumor growth in a subject having cancer comprising administering a therapeutically effective amount of a T cell genetically modified to express a CAR polypeptide comprising an antigen binding domain, a transmembrane domain, and an intracellular signaling domain, wherein the intracellular signaling domain comprises a variant CD32. In some aspects, any of the disclosed variant CD3ζ can be present in the CAR polypeptide.

In some aspects, the variant CD3ζ comprises two or more of ITAMs CD3ζa, CD3ζb, or CD3 ζc. In some aspects, the variant CD3ζ comprises two or more of ITAMs CD3ζa. In some aspects, the variant CD3 comprises two CD3ζa ITAMs and one CD3ζb ITAM, thus, the variant CD3 comprises CD3ζa, CD3ζa, CD3ζb. In some aspects, the variant CD3ζ comprises two CD3ζa ITAMs and one CD3 ζc ITAM, thus, the variant CD3ζ comprises CD3ζa, CD3ζa, CD3 ζc.

In some aspects, the variant CD3ζ comprises two or more of ITAMs CD3ζb. In some aspects, the variant CD3 comprises two CD3ζb ITAMs and one CD3ζa ITAM, thus the variant CD3ζ comprises CD3ζb, CD3ζb, CD3ζa. In some aspects, the variant CD3ζ comprises two CD3ζb ITAMs and one CD3ζc ITAM, thus, the variant CD3ζ comprises CD3ζb, CD3ζb, CD3ζc.

In some aspects, the variant CD3 comprises two or more of ITAMs CD3ζc. In some aspects, the variant CD3 comprises two CD3ζc ITAMs and one CD3ζa ITAM, thus, the variant CD3 comprises CD3ζc, CD3ζc, CD3ζa. In some aspects, the variant CD3ζ comprises two CD3 ζc ITAMs and one CD3ζb ITAM, thus, the variant CD3ζ comprises CD3ζc, CD3c, CD3ζb.

### I. Methods of Increasing Effector Function

Disclosed are methods of increasing effector function of a T cell comprising genetically modifying the T cell to express one or more of the CAR polypeptides disclosed herein, wherein the T cell has increased effector function.

Disclosed are methods of increasing effector function of a T cell comprising genetically modifying the T cell to express a CAR polypeptide comprising an antigen binding domain, a transmembrane domain, and an intracellular signaling domain, wherein the intracellular signaling domain comprises a variant CD3ζ. In some aspects, any of the disclosed variant CD3ζ can be present in the CAR polypeptide.

In some aspects, the variant CD3ζ comprises two or more of ITAMs CD3ζa, CD3ζb, or CD3ζc. In some aspects, the variant CD3 comprises two or more of ITAMs CD3ζa. In some aspects, the variant CD3ζcomprises two CD3ζa ITAMs and one CD3ζb ITAM, thus, the variant CD3 comprises CD3ζa, CD3ζa, CD3ζb. In some aspects, the variant CD3ζ comprises two CD3ζa ITAMs and one CD3 ζc ITAM, thus, the variant CD3ζ comprises CD3ζa, CD3ζa, CD3 ζc.

In some aspects, the variant CD3ζ comprises two or more of ITAMs CD3ζb. In some aspects, the variant CD3ζ comprises two CD3ζb ITAMs and one CD3ζa ITAM, thus the variant CD3ζ comprises CD3ζb, CD3ζb, CD3ζa. In some aspects, the variant CD3ζ comprises two CD3ζb ITAMs and one CD3c ITAM, thus, the variant CD3ζ comprises CD3ζb, CD3ζb, CD3 ζc.

In some aspects, the variant CD3ζ comprises two or more of ITAMs CD3ζc. In some aspects, the variant CD3 comprises two CD3ζc ITAMs and one CD3ζa ITAM, thus, the variant CD3ζ comprises CD3ζc, CD3c, CD3ζa. In some aspects, the variant CD3ζ comprises two CD3 ζc ITAMs and one CD3ζb ITAM, thus, the variant CD3ζ comprises CD3ζc, CD3ζc, CD3ζb.

J. Methods of Increasing Persistence of a T cell

Disclosed are methods of increasing persistence of a T cell comprising genetically modifying the T cell to express one or more CAR polypeptides disclosed herein, wherein the T cell has increased persistence.

Disclosed are methods of increasing persistence of a T cell comprising genetically modifying the T cell to express, a CAR polypeptide comprising an antigen binding domain, a transmembrane domain, and an intracellular signaling domain, wherein the intracellular signaling domain comprises a variant CD3ζ, wherein the T cell has increased persistence.

In some aspects, the variant CD3ζ comprises two or more of ITAMs CD3ζa, CD3ζb, or CD3c. In some aspects, the variant CD3ζ comprises two or more of ITAMs CD3ζa. In some aspects, the variant CD3ζcomprises two CD3ζa ITAMs and one CD3ζb ITAM, thus, the variant CD3 comprises CD3ζa, CD3ζa, CD3ζb. In some aspects, the variant CD3ζ comprises two CD3ζa ITAMs and one CD3c ITAM, thus, the variant CD3ζ comprises CD3ζa, CD3ζa, CD3 ζc.

In some aspects, the variant CD3ζ comprises two or more of ITAMs CD3ζb. In some aspects, the variant CD3ζ comprises two CD3ζb ITAMs and one CD3ζa ITAM, thus the variant CD3ζ comprises CD3ζb, CD3ζb, CD3ζa. In some aspects, the variant CD3ζ comprises two CD3ζb ITAMs and one CD3 ζc ITAM, thus, the variant CD3ζ comprises CD3ζb, CD3ζb, CD3 ζc.

In some aspects, the variant CD3ζ comprises two or more of ITAMs CD3 ζc. In some aspects, the variant CD3ζ comprises two CD3ζc ITAMs and one CD3ζa ITAM, thus, the variant CD3ζ comprises CD3ζc, CD3ζc, CD3ζa. In some aspects, the variant CD3ζ comprises two CD3 ζc ITAMs and one CD3ζb ITAM, thus, the variant CD3ζ comprises CD3ζc, CD3ζc, CD3ζb.

### K. Method of Increasing Bond Lifetime Between a T Cell and a Target Antigen

Disclosed are methods of increasing bond lifetime between a T cell and a target antigen comprising genetically modifying the T cell to express one or more of the CAR polypeptides disclosed herein, wherein the T cell and target antigen have an increased bond lifetime.

Disclosed are methods of increasing bond lifetime between a T cell and a target antigen comprising genetically modifying the T cell to express a CAR polypeptide comprising an antigen binding domain, a transmembrane domain, and an intracellular signaling domain, wherein the intracellular signaling domain comprises a variant CD3ζ, wherein the T cell and target antigen have an increased bond lifetime.

In some aspects, the variant CD3ζ comprises two or more of ITAMs CD3ζa, CD3ζb, or CD3 ζc. In some aspects, the variant CD3ζ comprises two or more of ITAMs CD3ζa. In some aspects, the variant CD3ζ comprises two CD3ζa ITAMs and one CD3ζb ITAM, thus, the variant CD3ζ comprises CD3ζa, CD3ζa, CD3ζb. In some aspects, the variant CD3ζ comprises two CD3ζa ITAMs and one CD3ζc ITAM, thus, the variant CD3ζ comprises CD3ζa, CD3ζa, CD3 ζc.

In some aspects, the variant CD3ζ comprises two or more of ITAMs CD3ζb. In some aspects, the variant CD3ζ comprises two CD3ζb ITAMs and one CD3ζa ITAM, thus the variant CD3ζ comprises CD3ζb, CD3ζb, CD3ζa. In some aspects, the variant CD3ζ comprises two CD3ζb ITAMs and one CD3ζc ITAM, thus, the variant CD34 comprises CD3ζb, CD3ζb, CD3 ζc.

In some aspects, the variant CD3ζ comprises two or more of ITAMs CD3c. In some aspects, the variant CD3ζ comprises two CD3ζc ITAMs and one CD3ζa ITAM, thus, the variant CD3ζ comprises CD3ζc, CD3ζc, CD3ζa. In some aspects, the variant CD3ζ comprises two CD3 ζc ITAMs and one CD3ζb ITAM, thus, the variant CD3ζ comprises CD3ζc, CD3ζc, CD3ζb.

L. Methods of increasing T cell receptor force in a T cell

In some aspects, T cell receptor (TCR) force is how strongly a TCR, or CAR acting as a TCR, binds to its target. In some aspects, a force of about 20 is considered a strong force.

Disclosed are methods of increasing T cell receptor force in a T cell comprising genetically modifying the T cell to express one or more of the CAR polypeptides disclosed herein, wherein the T cell has an increased TCR force at the CAR.

Disclosed are methods of increasing T cell receptor force in a T cell comprising genetically modifying the T cell to express a CAR polypeptide comprising an antigen binding domain, a transmembrane domain, and an intracellular signaling domain, wherein the intracellular signaling domain comprises a variant CD33, wherein the T cell has an increased TCR force at the CAR.

In some aspects, the disclosed variant CD3ζ ITAMs can attenuate or strengthen force of the TCR (or CAR) while the affinity for the antigen remains constant.

In some aspects, the variant CD3ζ comprises two or more of ITAMs CD3ζa, CD3ζb, or CD3 ζc. In some aspects, the variant CD3ζ comprises two or more of ITAMs CD3ζa. In some aspects, the variant CD3ζ comprises two CD3ζa ITAMs and one CD3ζb ITAM, thus, the variant CD3ζ comprises CD3ζa, CD3ζa, CD3ζb. In some aspects, the variant CD3ζ comprises two CD3ζa ITAMs and one CD3 ζc ITAM, thus, the variant CD3 comprises CD3ζa, CD3ζa, CD3 ζc.

In some aspects, the variant CD3ζ comprises two or more of ITAMs CD3ζb. In some aspects, the variant CD3 comprises two CD3ζb ITAMs and one CD3ζa ITAM, thus the variant CD3 ζcomprises CD3ζb, CD3ζb, CD3ζa. In some aspects, the variant CD3ζ comprises two CD3ζb ITAMs and one CD3 ζc ITAM, thus, the variant CD3ζ comprises CD3ζb, CD3ζb, CD3 ζc.

In some aspects, the variant CD3ζ comprises two or more of ITAMs CD3 ζc. In some aspects, the variant CD3 comprises two CD3ζc ITAMs and one CD3ζa ITAM, thus, the variant CD3ζ comprises CD3ζc, CD3ζc, CD3ζa. In some aspects, the variant CD3ζ comprises two CD3 ζc ITAMs and one CD3ζb ITAM, thus, the variant CD3ζ comprises CD3ζc, CD3ζc, CD3ζb.

M. Methods of Activating T cells

Disclosed are methods of activating a T cell expressing one or more of the CAR polypeptides disclosed herein comprising culturing the T cell with a cell expressing and detecting the presence or absence of IFN-γ after culturing, wherein the presence of IFN-γ indicates the activation of the T cell.

Disclosed are methods of activating a T cell expressing a CAR polypeptide comprising an antigen binding domain, a transmembrane domain, and an intracellular signaling domain, wherein the intracellular signaling domain comprises a variant CD3ζ comprising culturing the T cell with a cell expressing and detecting the presence or absence of IFN-γ after culturing, wherein the presence of IFN-γ indicates the activation of the T cell.

### N. Methods of Making Cells

Disclosed are methods of making a cell comprising transducing a cell with one or more of the disclosed vectors. In some instances, the cell can be, but is not limited to, T cells or NK cells. In some instances, the T cell can be a γδ T cell or an αβ T cell. In some aspects, the cell is a αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

Disclosed are methods of making a cell comprising transducing a T cell with one or more of the disclosed vectors. For example, disclosed are methods of making a cell comprising transducing a T cell with a vector comprising the nucleic acid sequence capable of encoding a disclosed CAR polypeptide to a subject in need thereof. Disclosed are methods of making a cell comprising transducing a T cell with a vector comprising the nucleic acid sequence capable of encoding a CAR polypeptide comprising an antigen binding domain, a transmembrane domain, and an intracellular signaling domain, wherein the intracellular signaling domain comprises a variant CD32.

### O. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the CAR polypeptides or nucleic acid sequences disclosed herein. In some aspects, disclosed are kits comprising one or more of the cells disclosed herein. Also disclosed are kits comprising one or more of the vectors disclosed herein.

### Examples

### A. Example 1

There is a current focus on tuning CAR intracellular signals via the ITAMs to increase effector function and persistence. Studies utilizing the CD28-CAR construct found that reducing the number of functional ITAMs from 3 to 1 (ITAM-AXX) allowed for better T cell persistence compared to the wild-type 3-ITAM zeta chain construct. Alternatively, switching out the entire 3-ITAM zeta chain domain with the 1-ITAM CD3 epsilon chain domain likewise increased T cell persistence but with reduced effector function. It is still unclear whether CAR T cells under stressed conditions (increasingly high tumor burden or low antigen density) may overwhelm these modified CARs. Optimization of CAR constructs are of high importance as current CAR therapies have significant drawbacks including CAR T cell toxicity from severe cytokine release syndrome, neurotoxicity and lack of CAR T cell persistence in vivo and expansion in vitro.

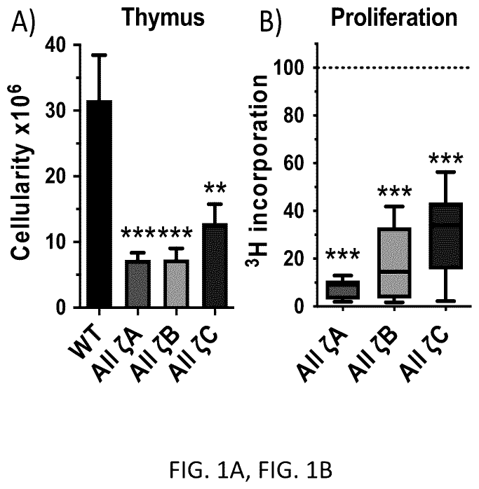

Each of the 3-zeta chain ITAM sequences (A, B and C) are unique with distinct kinetics of phosphorylation. Indeed, the 3 individual CD3 zeta chain ITAM motifs show a hierarchy of specificity for Zap-70, Shc, p85, Grb2 Fyn and Ras upon phosphorylation. Earlier studies utilizing CD3 retrogenic mouse technology indicates ITAM diversity is required for normal thymocyte development and function. In this system, each of the 10 unique ITAM sequences were replaced with a single ITAM sequence thereby maintaining 10 functional ITAMS of a single specificity. Only the ITAM sequence within each CD3 chain was modified. With this in vivo system, it was investigated whether certain ITAM sequences were necessary or redundant for activation of the TCR complex allowing thymocyte development and survival. Important for this proposal, utilizing identical CD3 zeta A, B or C ITAMs throughout the 10 ITAMs of the CD3 εδγζ chains had a significant impact on thymocyte survival (FIG. 1A). Correlating with low thymocyte numbers, the proliferation potential of peripheral T cells was severely impacted, indicating a signaling deficiency specific to cell cycle (FIG. 1B). Depending on ITAM usage, differential recruitment and phosphorylation of Zap-70 to zeta A, B and C ITAMs could be harnessed to tune CAR T cell signaling and survival. Importantly, ITAM diversity has also been shown to be necessary for TCR-driven proliferation and cytokine production. However, the role of CAR zeta chain ITAM diversity in CAR force signaling and function has not been fully explored.

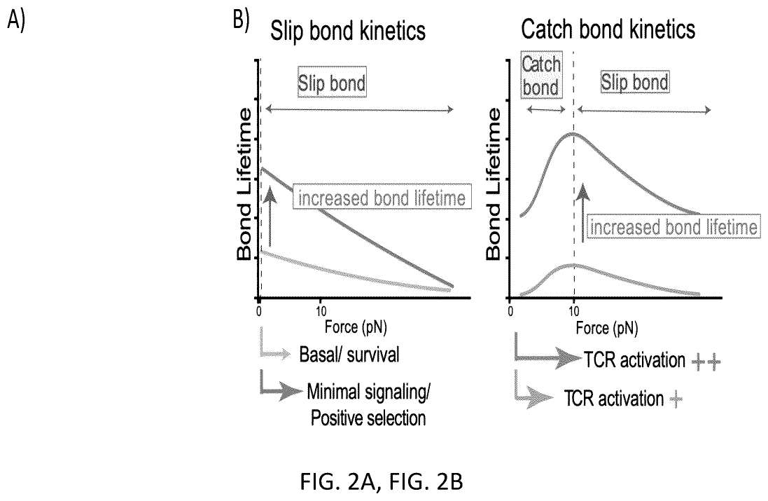

Interactions between TCR and pMHC occur at the interface of two cells in a two-dimensional (2D) membrane microenvironment. Initial binding events including affinity and kinetics of the TCR/pMHC interaction, as well as the CAR/protein epitope interaction, will dictate binding stability and downstream signaling events including phosphorylation of the ITAMs. Affinity provides the initial bond formation between TCR and pMHC which initiates signaling cascades. This contributes to a feedback loop with the cytoskeleton that applies increasing levels of force to the TCR/pMHC, and possibly the CAR/protein interaction, as a test of the quality of the antigen (FIG. 2A). Therefore, a CAR may also act as a mechanosensor similar to a TCR discriminating the quality of the interaction with antigen as cellular derived forces are applied to the bond. The 2D interaction between T cells and antigen presenting cells, both TCR/pMHC affinity and bond lifetime under force, is highly predictive of T cell function and fate. Engagement of the TCR/pMHC complex under force can result in one of two types of bonds. Increase bond lifetime as force is increased results in a “catch bond” which induces down-stream signaling. Alternatively, bond lifetimes that decrease as force increases are termed “slip bonds” and fail to lead to effector function (FIG. 2B, next page). T cell activation is often attributed to “optimal dwell-time” between the TCR/pMHC, but more recently TCR recognition and activation after encountering cognate antigen has been attributed to longer bond lifetimes that result from “catch bonds”. Lower affinity antagonist-pMHC interactions are for the most part the result of shorter bond lifetime, producing anything from a small catch bond to a slip bond. Ultimately, bond lifetime and force measurements provide us with an additional dimension to how T cell engages antigen for optimal response. Assays that make use of micropipettes or FRET have all been used to demonstrate that 2D affinity measurements of TCR provide a strong correlation between affinity and the extent of response. Importantly, it has been shown that bond lifetime under force superseded affinity and is required for different cellular outcomes. Stated another way, even high affinity T cells fail to optimally trigger functional responses if their TCR does not show catch bond formation. Now there is a tool to measure affinity, bond-lifetime, and force of the T cell/target interactions and these can be applied to in-depth analyses to determine the mechanodynamics of functional CARs.

The nature and establishment of these bonds are an active area of research as various TCR signalsome molecules such as CD45, Lck and Zap-70 may all play a role in the establishment of catch bonds and what amount of bond lifetime is necessary for a T cell. Another layer of complexity of bond lifetime for T cells is that as a T cell is triggered and the ITAMs become phosphorylated, the signaling cascade applies additional force to the bond (inside-out applied force). By fine-tuning the combination of ITAM sequences and, as a result, reduce or increase ITAM dependent signaling, force and bond lifetime between the CAR and CD19 can be modulated. This can allow one to determine optimal force, proliferation, survival, and phenotypic fate.

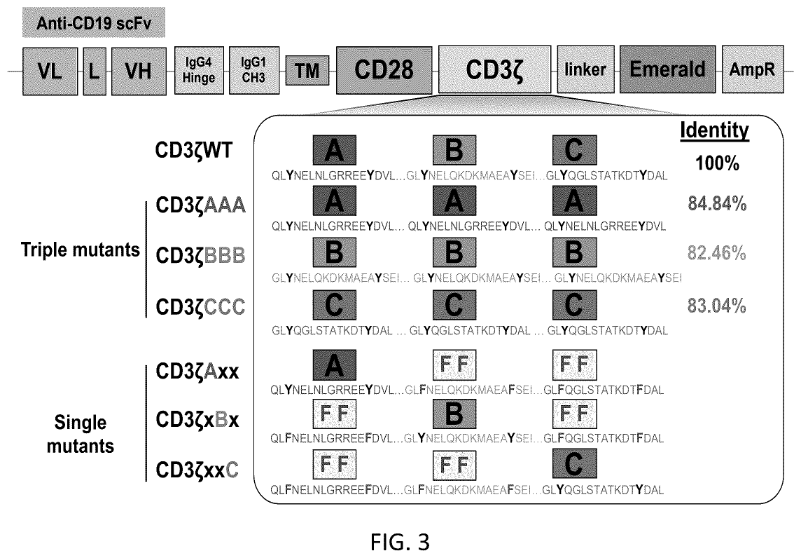

To address the role of CD3 zeta ITAM diversity in Chimeric Antigen Receptor signaling and bond lifetime, a well-characterized second-generation mouse (m) CD19 CAR construct (1D3) and a clinically approved second generation human (h) CD19 CAR construct (FMC63) was used (FIG. 3). The advantage of using CD19 CAR, is that the construct has been used in many CAR optimization studies, and allows us to directly compare these modifications to what has been previously observed. Recent studies have inhibited CD19 CAR ubiquitination, reduced functional ITAMS, or have utilized an entirely different CD3 chain (CD3 epsilon), all of which have increased antitumor potency. In each of the systems, calibration of CAR activation led to memory T cell differentiation (reduced ITAMS and reduced ubiquitination or stronger AKT signaling (CD3 epsilon chain). finetuning the ITAM sequences can also help deliver lower signal transduction, resulting in less cytokine release and increase memory CAR T cell development. Conversely, it is also possible that restricting ITAM diversity (i.e., zeta ITAM-AAA) can increase Zap-70 binding at all three ITAMs or increase AKT signaling and signal transduction.

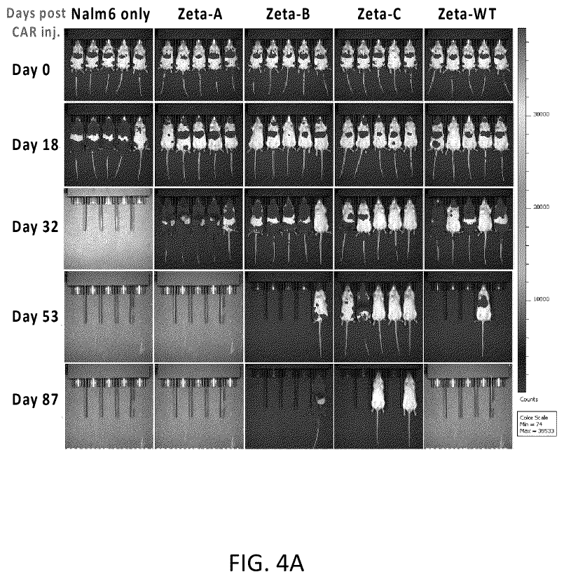

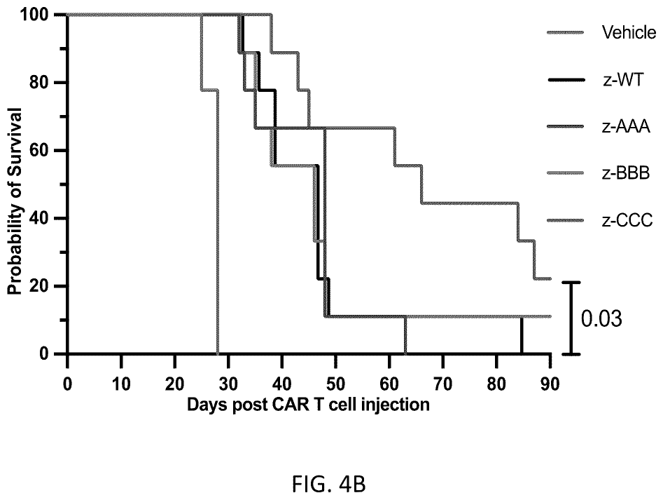

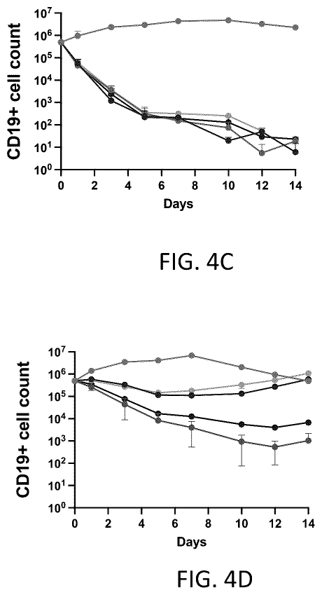

First, it was investigated whether modifying the sequence of the ITAMs but keeping the same number could alter tumor clearance. To do this, human CD19 WT-zeta ITAM reactive CAR T cells were compared with the z-AAA, z-BBB and z-CCC CD19 ITAM mutants in response to the CD19+Nalm-6 acute lymphoblastic leukemia (ALL) tumor cell line in the NOD.RAG−/−. Gamma−/−(NRG) mouse model. Following luminescence and mortality of each group over time, it was found that the ITAM z-CCC group controlled tumor growth significantly better than the ITAM WT group and the other ITAM mutant groups out to 90 days (FIG. 4). These data indicate ITAM sequence as well as number may have a significant role in CAR signaling. Therefore, how maintaining the number of Zeta ITAMs (3) but modifying their sequences will result in a calibration of CAR signaling and alter effector function is determined.

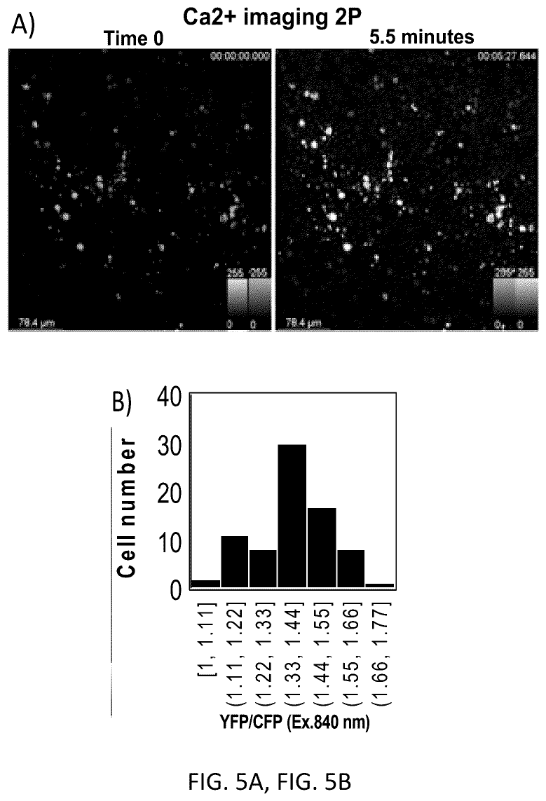

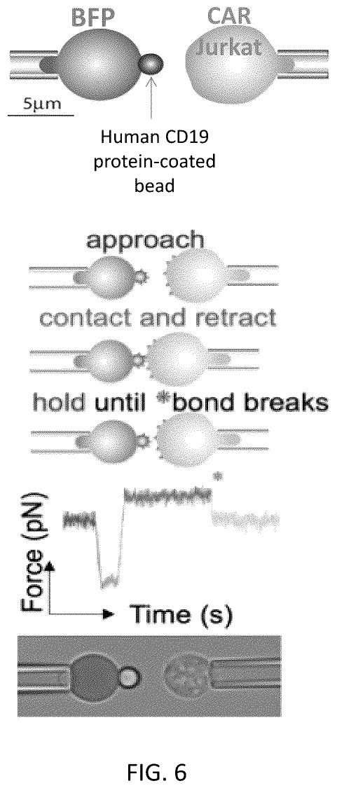

While it is clear that CAR ITAMs are responsible for transducing extracellular stimuli into intracellular fate decisions, a role for individual zeta ITAMs in tuning tumor specific responses is just emerging. First, CAR constructs were developed with ITAM sequences that were replaced with a single ITAM sequence (the remainder of the zeta chain remains intact) that can be transduced into mouse and human T cells (FIG. 3). At the amino acid level, the WT zeta ITAM sequence is only 84% similar to that of the AAA ITAM sequence, 82% of BBB and 83% of CCC. Comparing AAA to BBB and CCC to each other, lowers similarity to 76-78% (FIG. 3 and not shown). Using this approach, individual ITAMs were altered and various combinations were expressed to assess ITAM sequence and multiplicity. Second, to address in vivo and ex vivo signaling of CAR ITAM mutants without the treatment of a calcium dye, a Ca2+ biosensor transgenic mouse was generated with Foster/fluorescence resonance energy transfer (FRET)-based Ca2+ indicator yellow-cameleon 3.60 (YC3.60) (FIG. 5) similar to already published mice. When expressed within CAR T cells, in vivo (or during Biomembrane Force Probe measurements (BFP)) imaging can allow for real-time receptor mediated signaling by CAR and CAR-ITAM mutants. Third, CAR interactions can be measured with protein (CD19) by the use of Biomembrane Force Probe (BFP). To provide better understanding of the dynamic nature of T cells and the applied forces to protein: protein bonds between cells, a diagram of the BFP is shown in FIG. 6. A streptavidin-Maleimide bound glass bead coated with pMHC of interest is attached to the apex of the RBC (left micropipette). For the human CAR T cell system, beads coated with human CD19 are attached to RBCs. After contact is made, the RBC is retracted, and the pull from the T cell is measured as force over time. At a certain point, the bond breaks, and the force dissipates. Using this innovative method by coating an RBC/bead with CD19 protein, one can understand how ITAM diversity dictates force and regulates CD19 specific CAR dependent intracellular signaling. As a control, beads are coated with no ligand to assess specificity. Finally, whole cell phosphoproteomics can be performed by heavy labeling of CAR T cells to determine whether ITAM restriction differentially phosphorylates signal transduction pathways and whether ITAM diversity contributes to the generation of inside-out force.

### 1. Results:

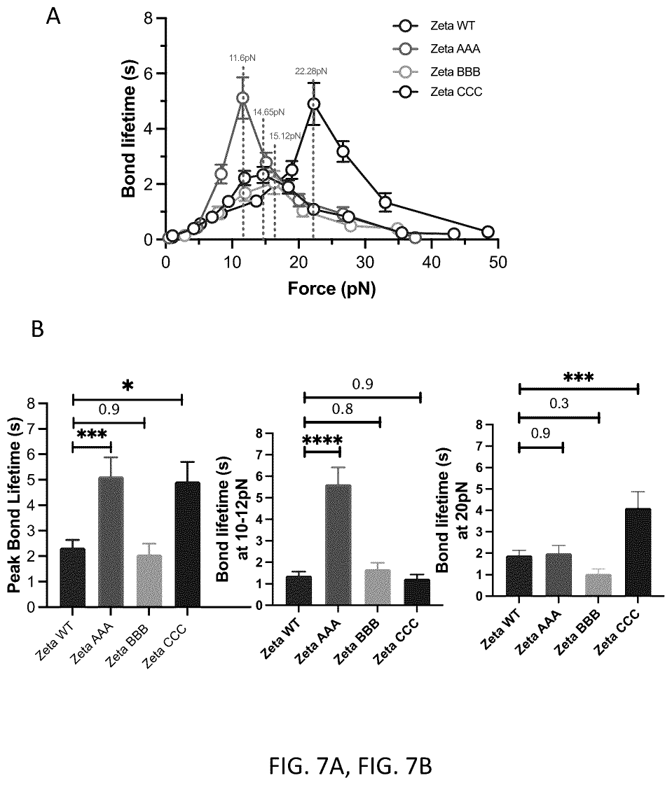

One possible mechanism as to how ITAMs could be a mechanosensor for TCR signaling is the role for Zap-70 in the establishment of catch bonds. As a T cell is triggered and the ITAMs become phosphorylated by Lck which recruits Zap-70, the signaling cascade applies additional force to the bond (inside-out applied force). If ITAM phosphorylation and Zap-70 recruitment is altered between each CAR ITAM mutant, biomembrane force may also be impacted. Whether force was altered in Jurkat cells expressing CD19 specific CAR ITAM mutants (2-AAA, z-BBB, z-CCC or WT zeta ITAMs) was tested. Three distinct force and bond lifetime measurements between zeta-AAA, zeta-BBB and zeta-CCC ITAM mutants were seen (FIG. 7A). The zeta-AAA ITAM (Red) mutant CAR had the longest bond lifetime of 5.6 sec compared to the WT ITAM CAR (2 sec), the zeta-BBB (Green) 2 sec and the zeta-CCC (Bluc) 4.1 sec ITAM mutants (FIG. 7B). The amount of bond lifetime seen with zeta-AAA is indicative of more cell activation and effector function in T cells but it is unclear the impact on CAR T cell function. Additionally, and perhaps more importantly, the zeta-CCC (Bluc) ITAM mutant CAR displayed the highest peak force of 20pN, compared to all other CARs (at slightly lower bond lifetime compared to z-CCC mutant) may display decreased signaling, activation and effector function leading to CAR T cell persistence. These results are important for a number of reasons. First, it was presumed that CARs would display higher force and bond lifetime than a TCR due to the antibody/antigen interaction. This is not the case and surprisingly CAR and TCR force measurements are similar perhaps due to zeta chain initiated signal transduction pathways. Second, the data indicates specific ITAMs can attenuate or strengthen force while the affinity for the antigen remains constant. This has important implications in “tuning” the activation of a CAR T cell without changing specificity.

Signaling and mass spectrometry phosphoproteomics can be investigated as well as studying functional consequences of the human CAR ITAM mutants in NOD.RAG−/−. Gamma−/−(NRG) mice. Two long-term survival studies have been performed comparing all human CAR CD19 ITAM mutants in response to the CD19+Nalm-6 acute lymphoblastic leukemia (ALL) tumor cell line in vivo (FIG. 4). CD19 ITAM-CCC group controls tumor growth significantly better than the ITAM WT group and other ITAM mutant groups out to 90 days. This matches the prediction from the BFP measurements that the z-CCC ITAMS may allow for better long-term fitness and effector function. Both TMT and SILAC labeling coupled to highly-selective, wide-scale phosphoenrichment strategies utilizing either pTyr superbinder and TiO2 in the analysis of T cell signaling by mass spectrometry have been used. This technology recently revealed unique T cell signaling pathways in CAR T Raji cocultures not seen previously with MHC-tetramer OT1 stimulations. In this manner the phosphoproteome of each ITAM mutant CAR expressed within primary human T cells can be compared with Jurkat cells after coculture with CD19 expressing cells. These data can lead to detailed analysis of basal, early and late signaling pathways after cell to cell contact with cognate CAR tumor antigen.

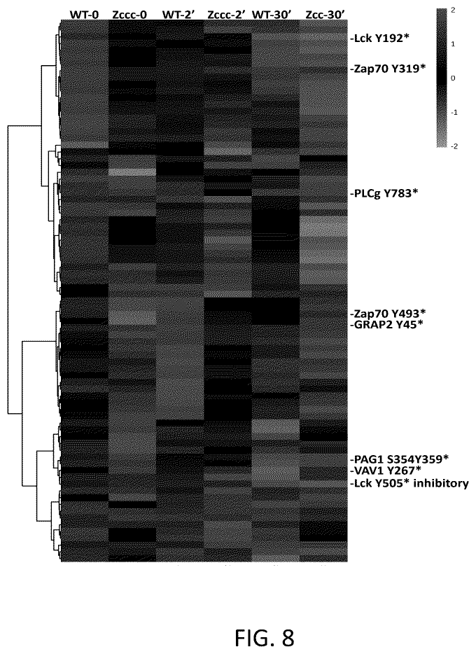

Phosphoproteomic studies have been performed with CAR expressing Jurkat cells indicating an increased activation (at basal, 2 min and 30 min) of WT CAR T cells compared to Zeta CCC mutant CAR T cells. (FIG. 8). The data implies WT CAR T cells are at a higher basal and initial activation state compared to ITAM mutant z-CCC. z-CCC also appears to have higher basal level of phosphorylation of the Lck inhibitory tyrosine, Y505, VAV1 and Csk associated molecule PAG1, while phosphorylation of Zap70, Grap2, PLCg1 and Lck is consistently found to be higher within the WT CAR group at 0 and 2 minutes after Raji stimulation.

### 2. Determine Whether CD3 Zeta ITAM Motifs are Necessary or Redundant for Optimal CAR Signaling.

After Lck recruitment, the kinetics of phosphorylation for each of the individual zeta chain ITAM tyrosine residues in positions A, B and C of the TCR complex appear to be distinct, with ITAM-C exhibiting the slowest phosphorylation events. However, the role of CAR zeta chain ITAM diversity in generating intracellular signaling necessary for optimal CAR T cell effector function and persistence is not fully understood.

i. Research Design and Methods:
a. Quantify and Compare Early and Late Intracellular Signaling Events Between Zeta WT, z-AAA, z-BBB and z-CCC Mouse and Human Mutant CD19 Reactive CARs by Flow Cytometry, Western Blot and I.P.

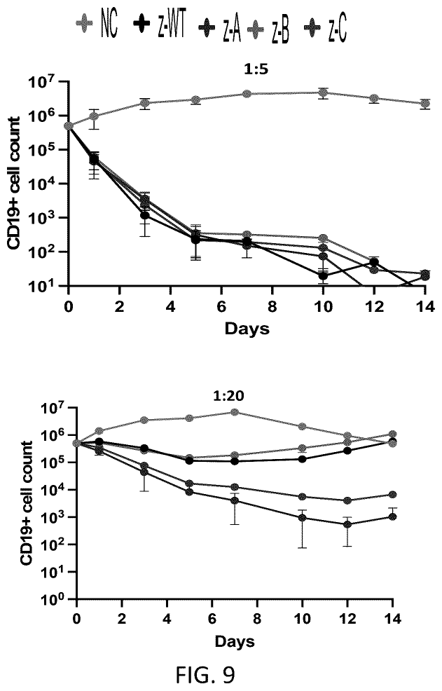

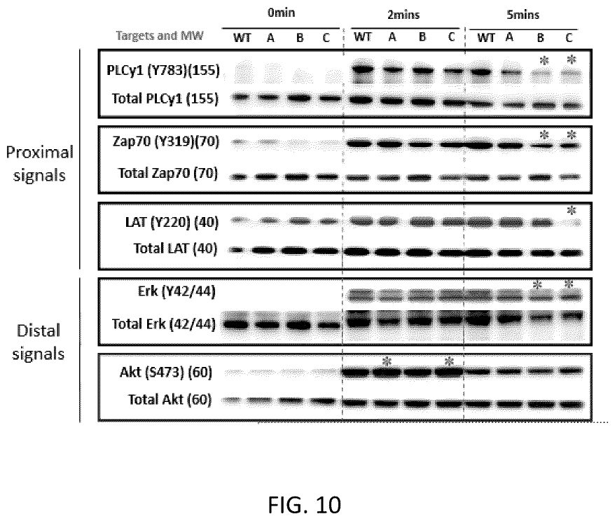

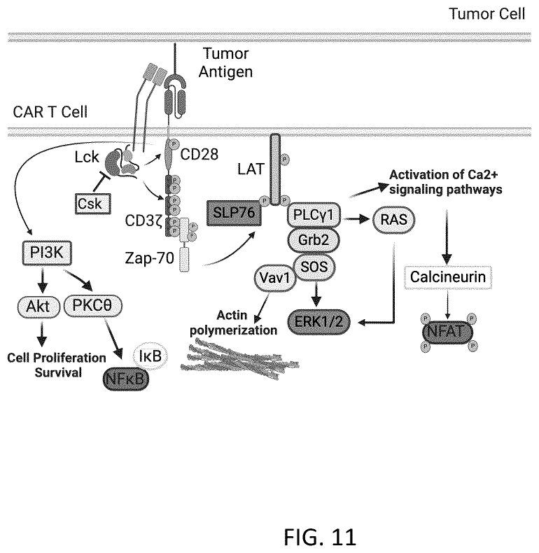

Western Blot of the CAR complex. Maintaining the number of zeta ITAMs (3) but modifying their sequences can improve T cell persistence and memory T cell formation without the loss of cytolytic function. All human and mouse primary CAR mutant ITAM T cells equally express the CAR and can kill tumor cells in vitro comparable to WT zeta ITAM CAR T cells at the highest E: T ratio (FIG. 9). However, as the CAR T cells are titrated down, the z-CCC and z-AAA ITAM mutant CAR T cells appear to have an advantage. Early and late CAR mediated signaling can be interrogated to better understand whether other functional outcomes such as cytokine production, survival and persistence can be impacted. Utilizing primary CD8+human T cells transduced with FMC6 anti-CD19 CAR ITAM mutants and stimulated with CD19+Raji cells (or unstimulated) a reduction in proximal signaling (Zap70 and PLCα1) of the Zeta ITAM mutants was observed and in particular the z-CCC and z-BBB mutants at resting and 5 minutes (FIG. 10) indicating ITAM dependent signal attenuation. In vitro: This experiment can be repeated with both CD4+ and CD8+mouse primary and human primary CAR T cells with additional timepoints out to 40 minutes to observe persistence. Including Zap-70 and PLCα1, phosphorylation of other proteins including negative regulators CSK and SHP1/2 as well as signal transducers, AKT, LAT associated proteins SLP76 and Map Kinase ERK, that feed into cell proliferation, cytoskeletal rearrangement and cytokine function respectively can be measured (FIG. 11). In order to interrogate phosphorylation and protein interactions specific to the CAR as opposed to the whole cell, I.P. western blots can be performed of the CAR zeta chains (myc-tagged) from both primary human and mouse CAR T cells (CD4+ and CD8+). zeta-ITAM phophorylation, Lck, SLP76, Zap-70, and PLCαphosphorylation can be probed for. Compared to the wild-type zeta ITAM, z-CCC can recruit less proximal molecules (SLP76, Zap-70 and PLCα-1), which can result in lower CAR mediated signaling over time. LAT and PLCα-1 dependent signaling pathways by Western blot (distal proteins) and I.P. western blot analysis after CAR crosslinking and CD19 coated beads. Additionally, functional readouts of attenuated or increased CAR signaling such as T cell proliferation and cytokine production (IFNγ, TNFα, Perforin and IL-2) can be measured at 0, 24, 48 and 72 hours after stimulation with CD19+tumor cells (Eμ-ALL01-mouse or Nalm-6-human). In vitro persistence assays may give an indication of in vivo potential. Next, proliferation/fold expansion can be measured after tumor stimulation at day 0, then restimulate at 7 and 14 days. The ITAM mutant CAR T cells can be characterized at each timepoint to ascertain whether there is an indication of early upregulation of exhaustion markers including memory-cell like markers CD44, CD62L, CD45RA, apoptosis marker Annexin V+ and exhaustion markers PD-1, Tim3, Lag3 by flow cytometry. Each study can be repeated with patient tumor isolates of varying (low) CD19 expression to test antigen sensitivity of each CAR mutant. Finally, mitochondrial respiration and glycolysis (Seahorse) can be measured as it has been shown that there are distinct metabolic programs within CAR T cells between the use of 41BB (persistence) compared to CD28 co-stimulatory (high activation) domains. It is possible that z-CCC can persist after re-challenge and can be sensitive to lower CD19 expression compared to WT or z-AAA due to its lower basal and lower activation. Although ITAM usage has not been correlated with metabolism to date, higher effector function would correlate with higher glycolytic metabolism.

b. In Vitro:

Utilizing Phosphoproteomics, determine the relative quantitation of proteins and phosphoproteins in human ITAM mutant CD19 reactive CAR CD4+ and CD8+ T cells. Basal phosphorylation of the CD3 zeta chain within the TCR58 and the CAR occurs naturally. In the case of basal CAR signaling this can be detrimental to the efficacy of CAR T cell survival due in part with endogenous Lck association. Therefore, negative feedback mechanisms are needed to control unwarranted activation. After CAR ligation and recognition of tumor antigen, phosphorylation occurs rapidly. However, recognition and binding of tumor antigen by an scFV of the CAR will have different kinetics in activation and deactivation compared to a TCR. ITAM CAR mutants phosphorylate upstream and downstream signaling molecules such as Zap-70 and PLCα-1 differentially and generate drastically different force measurements depending on the ITAM present (FIGS. 7, 8 and 10). This can lead to differential phosphorylation of proteins globally between each mutant and may impact the signaling pathways responsible for CAR T cell death, persistence and effector function (FIG. 11).

With the SILAC/TMT phosphoproteomic strategy, the relative quantitation of proteins and phosphoproteins can be determined in human ITAM mutant CD19 reactive CAR CD4+ and CD8+ T cells after CAR specific stimulation (early and late signaling) and wide-scale total phosphorylation measured in the basal state. SILAC arg/lys heavy-labelled D19+Raji cells can be cocultured with each light arg/lys labelled ITAM CAR mutant, differentiating the protcomes, while TMT will be used to increase accuracy and precision while reducing the amount of T cells needed for the experiment ten-fold while maintaining a sequencing depth of >2,000 unique pY sites in primary T cells. Using SILAC, 99.5% of all proteins in Raji incorporate the heavy arg/lys which provides confident quantitation of T cell phosphorylation without interference from Raji B cell phosphorylation in the cocultures. There are computational tools for the aggregation, visualization, statistical analysis, and pathway prediction of T cell signaling networks from wide-scale proteomic analyses. These tools facilitate extraction of statistically meaningful observations to enable the elucidation of the structure of regulatory networks. From these studies one can determine the relative quantitation of global proteins and phosphoprotein levels in mouse and human ITAM mutant CD19 reactive CAR T cells that may lead to alterations with inside out force generation and effector functions.

Prediction of protein networks is essential to assemble protein interaction data into testable models. PeptideDepot software now integrates new phosphorylation site-specific ontologies created from scraping SCANSITE, PhosphoNET, and the Human Protein Reference Database as well as traditional protein function ontologies such as GO and KEGG. A protein set enrichment analysis (PSEA) tool has been created for detection of significant enrichment of these ontology terms within large proteomic datasets similar to the GSEA tool that is commonly applied to genomic data. PSEA can be applied to the proteomic data collected here to check for significant enrichment of protein classes, signaling functions, signaling domains or upstream kinases or phosphatases for ITAM specific signaling circuits. At resting and after stimulation with CD19+Raji Cells, the z-CCC mutant CAR Jurkat cells have less TCR signaling phosphorylated peptides including Zap70, Lck and PLCα-1 among a list of others (FIG. 8) as well as significant alterations in phosphorylation of proteins associated changes in Metabolism and IL-2 signaling (data not shown).

3. Determine Whether ITAM Diversity Alters Force and Bond Lifetime with ITAM Restricted CARs.

The TCR and pMHC forms a trimolecular complex at the cell surface in a two-dimensional (2D) membrane microenvironment. Initial binding events including affinity and dwell time of the TCR/pMHC interaction dictate binding stability, ITAM phosphorylation and downstream signaling events. The 3 individual CD3 zeta chain ITAM motifs show a hierarchy of specificity for Lck recruitment and subsequent Zap-70 upon phosphorylation while modifying the ITAM usage alters TCR signaling. Restricting ITAM diversity can differentially regulate bond lifetime allowing formation of a catch bond resulting in varying effector function. Data utilizing Jurkat and primary Human T cells indicates that zeta CCC-ITAM usage allows for lower basal and CAR induced pZap70 and PLCα-1 signaling early after CAR ligation, while z-AAA CARs appear similar to WT-ITAM CAR and and z-BBB signaling can be decreased (FIGS. 10 and 7). Interestingly, when bond lifetime of Jurkat cells is measured, z-BBB ITAM is similar in peak force and bond lifetime compared to the WT zeta ITAM, while z-CCC shows higher peak force and bond lifetime. This indicates a possible role for ITAM usage in force generation from an inside out mechanism(s) (FIG. 2). Force/Bond lifetime data with Jurkat cells also indicates WT CARs and ITAM mutant CARs form catch bonds but with varying measurements that could be predictive of function and persistence (FIG. 7). Whether ITAM restriction results in a catch or slip bond can be determined utilizing primary Human and Mouse CAR T cells. ITAM mutant CD19 reactive CARs can be tested in the micropipette adhesion frequency assay to measure affinity and the BFP assay to determine peak bond lifetime and amount of force. The restricted zeta ITAM CARs can maintain affinity, but vary in their force in order to provide a range in recognition of CD19. z-CCC ITAM mouse and human CAR T cells can maintain affinity but can have higher bond lifetime and peak force similar to the Jurkat cell data, allowing for differential recruitment and phosphorylation of Zap-70, Lat and other components of the CAR signalsome.

i. Research Design and Methods:
a. Determine the Amount of Force that Human and Mouse CD19 Reactive CAR T Cells Generate Compared to Single ITAM Usage CARs (z-AAA, z-BBB and z-CCC).

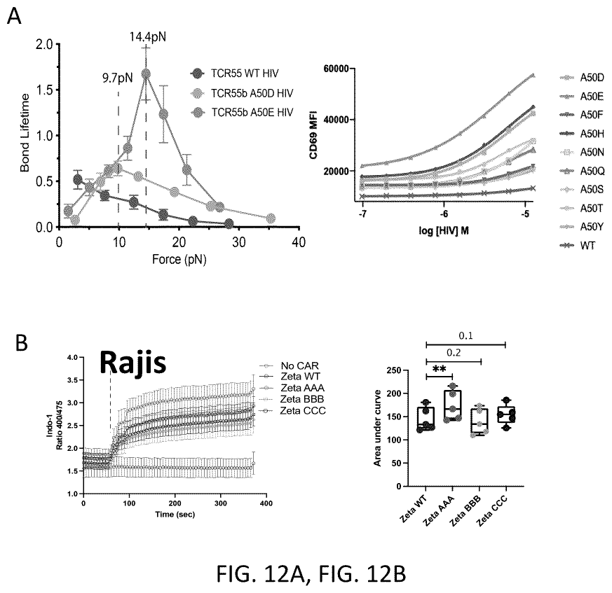

Mechanosensing of CD19 CAR is intricate and characterized by different parameters whose relative contribution is poorly understood. Force measurements in Jurkat cells expressing CD19 specific CARs is the first to try to uncouple force from affinity. CD19 specific CARs in human PBMC derived CD4+ and CD8+ T cells (de-identified from ARUP laboratories) and mouse primary CD4+ and CD8+ T cells with WT ITAMs, ITAM z-AAA, ITAM z-BBB and ITAM z-CCC can be investigated to determine the significance of both peak bond lifetime and force magnitude in relation do downstream signaling. HIV specific TCR (TCR55) with mutations in the CDR2 region can elicit changes from a slip bond (0pN) to a catch bond (˜10pN) (FIG. 12, Left) and good CD69 expression to a longer/stronger catch bond (˜15pN) (FIG. 12, Right). Hence, TCR CDR2 mutations with increased peak bond lifetime corelate with increased CD69 expression (FIG. 12 Right). Peak bond lifetime of primary human and mouse CD19 CAR CD4+ and CD8+ T cells can be compared with early activation markers such as CD69 and Nur77. The B6. YC3.60 calcium reporter mouse strain can be used to generate mouse CAR ITAM mutant T cells for in vitro and in vivo (Spleen) use in the presence of CD19+tumor cells (mouse e-mu). Ca+flux pre-injection can be tested and Force and real-time Ca+Flux measured. For human CAR T cells, the Fura-2 Calcium indicator can be used. If CAR ITAM mutant peak force or bond lifetime correlates with function, BFP may be a tractable method to screen efficacy of future CARs before expensive in vivo and in vitro models are tested. To this end the ITAMs of a human CAR can be used in the clinic at Huntsman Cancer institute (BCMA-Multiple Myeloma) and measure force to compare the CD19 and multiple myeloma BCMA specific CAR ITAM mutants.

b. Determine Whether Basal Recruitment and Activation of Zap-70 Alters Slip/Catch Bond Lifetime.

ITAM specific phosphorylation allows for the differential force measurements observed in FIG. 7. After Lck recruitment, the kinetics of phosphorylation for each of the individual zeta chain ITAM tyrosine residues in positions A, B and C of the TCR complex appear to be distinct, with ITAM-C exhibiting the slowest phosphorylation events. Therefore, inhibiting Zap-70 should inhibit CD19 specific-CAR ITAM phosphorylation after CD19 ligation. In this manner, force generated in the absence or reduction of Zap-70 and ITAM phosphorylation can be measured. Utilizing Zap-70 KO and Zap-70 analog sensitive (AS) Jurkat cells lines one can interrogate the role of ITAM phosphorylation in generating Force. The Zap-70AS (Analog Sensitive) mutant utilizes a genetically selective inhibitor specific to position 414 to accommodate a bulky 3-MB-PPI analog. Therefore, using Zap-70AS Jurkat cells expressing each mutant CAR ITAM, the inhibitor can be titrated and determine whether Force decreases with Zap-70 inhibition and whether there will be a change between catch/clip bond formation.

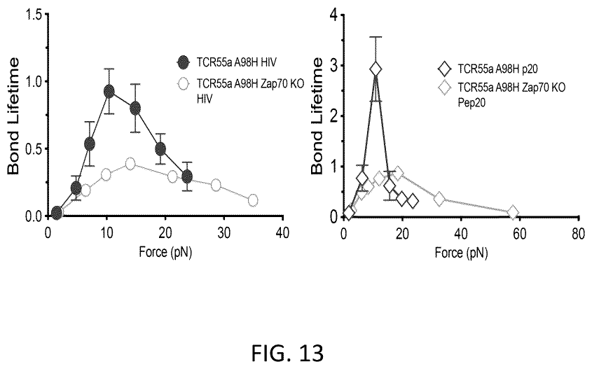

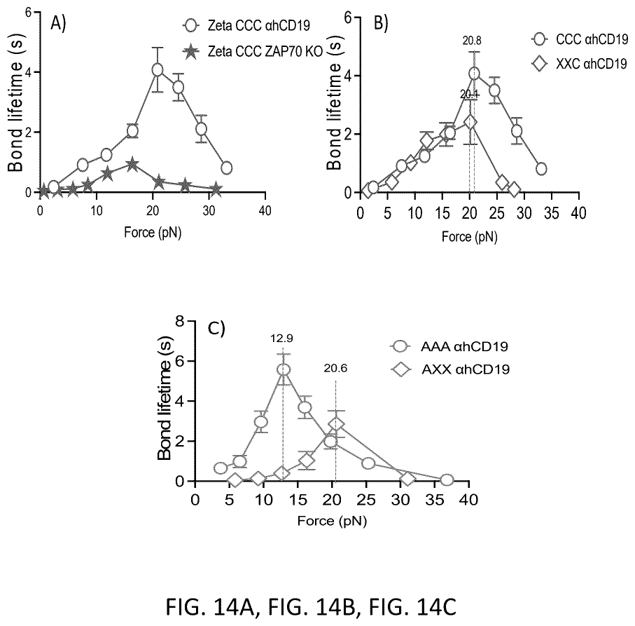

Testing one of the modified human HIV TCRs, a single amino acid substitution was found in the CDR3 region at A98H elicits good signaling when activated when compared it to the TCR55 A98H ZAP-70 KO (FIG. 13). Significant decreases were seen in bond lifetime from ˜0.9 sec in the WT (shaded circles) to ˜0.3 sec in the ZAP-70 KO (open circles) using cognate pMHC. Interestingly, using the pep20 pMHC which induces stronger signaling a drop is seen from 2.9 sec in the WT (blue diamonds) to ˜0.8 sec in the ZAP-70 KO (grey diamonds). This decrease in peak bond lifetime correlated with decreases in T cell functionality and activation. This data demonstrates the feasibility and sensitivity of the BFP assay and the contribution of ZAP-70 in “tuning” bond lifetime under force which are critical for informing T cell function. Furthermore, these data demonstrate the internal machinery of a T cell impacts external receptor bond lifetimes. These findings indicate that there is limited understanding in deducing receptor/ligand bond lifetime apart from cell surface molecule expression. CD19 specific CAR can be used to investigate early recruitment of Zap-70 in CAR ITAM phosphorylation as it relates to inside-out force after CD19 ligation. Bond lifetime and peak force of the zeta-ITAM mutants was compared with the Zap-70 KO Jurkat cell lines. All mutants showed a dramatic shift in bond lifetime similar to the SKW3 T cell cline (FIG. 14A), however, a noticeable decrease in force needed for peak bond lifetime was observed with the z-CCC mutant. Previously published single-Zeta ITAM mutants z-XXC and z-AXX also demonstrated an increase in tumor clearance and survival compared to WT ITAM CD19 specific CAR T cells. The z-AAA and z-CCC mutants were compared to the published z-AXX and z-XXC mutants to determine whether reducing functional ITAM number altered force. Interestingly, both the z-XXC and z-AXX pulled higher force at peak bond lifetime albeit with lower bond lifetime compared both z-AAA and z-CCC, indicating ITAM number may determine bond lifetime while the ITAM sequence may determine peak force (FIG. 14B and C). Zap-70AS Jurkat line can be used to “dial down” Zap-70 activity to better understand basal ITAM phosphorylation and Zap-70 activity between each zeta ITAM mutant. An important observation is whether there is an amount of Zap-70 activation after a specific interaction (CD19/CAR) that allows the change from a catch bond to a slip bond, and in the absence of Zap-70, whether Vav/WASP recruitment and phosphorylation is altered.

### 4. Determine the Role of Individual CD3Zeta ITAM Motifs in Persistence and Function of CAR T Cells.

Data indicate the number and sequence of functional CAR ITAMs directs downstream signaling and T cell fate (memory). Additionally, the type of CD3 chain associated with the CAR construct (epsilon versus zeta), impacts signaling and memory T cell formation. Therefore, maintaining the number of zeta ITAMs (3) but modifying their sequences can improve CAR T cell persistence and memory.

i. Research Design and Methods:
a. Determine the Functionality and Efficacy of Tumor Infiltrating CD19 Specific Zeta Chain Mutant CAR T Cells.

Human ITAM mutants (A, B and C) CAR T cells have varying killing potential to that of WT ITAM CAR T cells (FIGS. 4 and 9). Mouse CAR T cells: To test tumoricidal activity, various in vitro experiments can be performed alongside in vivo experiments. The B-ALL (Eμ-ALL01) CD19+tumor cell line can be used in both settings. Resting primary C57BL.6 CD4+ and CD8+CAR T cells can be cocultured with CD19+Eμ-ALL01 cells at effector: target (E: T) ratios of 4:1, 2:1, 1:1, 1:2, and 1:4 and measure CD19+killing and T cell activation. Control cultures of empty vector CARs and CD19+Eμ-ALL01 cells and CAR T cells only will be included. At varied timepoints of 24, 48, 64 and 72 hours, cells from all cultures can be isolated and stained with anti-mouse antibodies for cell identification CD3, CD4, CD8, CD19; for T cell activation CD69, CD25, CD62L, CD44, PD1, CD103, OX40, Ki67; for transcription factors Tbet, Blimp1, Eomes, Bcl6; for cytotoxic molecules Granzyme B, perforin; and analyzed by flow cytometry. The total counts of CD19+ cells after coculture can be measured. Further, supernatants from cocultures can be analyzed by ELISA for cytokines including IFNγ, TNFα, IL2. Results from empty vector “CAR” T cells only can be used as controls to exclude spontaneous cytokine release. These data can be repeated for CD4+ T cells. Human CAR T cells: To test more stringently the tumoricidal activity of ITAM mutant human CAR T cells, resting primary CD4+ and CD8+CAR T cells can be cocultured with CD19+Nalm6 cells at effector: target (E: T) ratios of 4:1, 2:1, 1:1, 1:2, and 1:4. At varied timepoints of 24, 48, 64 and 72 hours, cells from all cultures will be isolated and stained with anti-human antibodies for cell identification CD3, CD4, CD8, CD19; for T cell activation CD69, CD25, CD45RO; for proliferation Ki67; for transcription factors Tbet, Blimp1, Eomes, Bcl6; for secretion marker CD107a; for cytotoxic molecules Granzyme B, perforin; and analyzed by flow cytometry. The total counts of CD19+ cells after coculture can be measured for killing efficiency. Further, supernatants from cocultures can be analyzed by ELISA for cytokines IFNγ, TNFα, IL2. Results from empty vector CARs and CAR T cells only can be used as controls to exclude spontancous cytokine release. Human CD19+tumor isolates (HCl biobank) with varying CD19 expression can be tested to determine each CART ITAM mutant sensitivity. ITAM mutants can be generated for the BCMA CAR specificity and test in vitro function as described above.

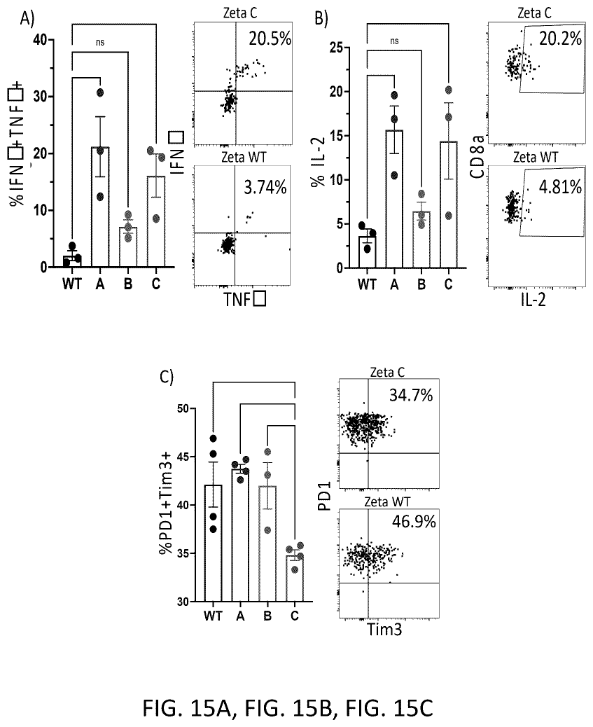

Finally, the mouse and human ITAM mutant CAR T cells can be tested in vivo. In this first part (mouse CAR) of the aim the immunocompromised C57BL/6 RAG1−/−mouse model for B-ALL (Eμ-ALL01) can be used. To compare short term effector function, luc-Eμ-ALL01 cells can be injected, wait 3 days, then inject CAR T cells. After 1 week, bone marrow, spleen and peripheral lymph nodes are isolated to measure cytokine production and activation markers described above for each ITAM mutant CAR T cell group. z-AAA and z-CCC mutant ITAM CAR T cells displayed the highest relative ex vivo cytokine response for IFNα/TNFγ and IL-2 production (FIG. 15A and B). Importantly, only z-CCC had a significantly reduced level of PD1/Tim3 indicating a less exhausted phenotype (FIG. 15C). Increased bond lifetime at peak force (FIG. 7) could be indicative of greater effector function but less survival while lower bond lifetime at peak force may allow for a more functional (less exhaustive) phenotype. Draining lymph nodes have been shown to harbor a subset of dendritic cells that maintain a reservoir of memory like tumor specific T cells that correlate with tumor clearance. These experiments can be repeated in an immunocompetent C57BL/6 WT mouse setting in order to evaluate CAR T cell functionality in the presence of endogenous B cells and other immune cells.

Human CAR: NRG mice can be used for in vivo cytotoxicity assays with human CAR mutant ITAM T cells. At Day-3, 2.5×10⁵ luciferase-GFP expressing Nalm6 (luc-GFP Nalm6) cells will be inoculated intravenously into the mice. Mice can be separated into five groups. At day 1, a total of 1×10⁶ CD4+ (20%)/CD8+ (80%) primary ITAM mutant or WT ITAM CAR T cells will be injected intravenously into mice. No CAR-expressing T cells will serve as control. The results from FIG. 4 can be confirmed for tumor burden and survival evaluated by bioluminescence imaging using the in vivo imaging system (IVIS) and images analyzed with the Living Image software. Health of the mice can also be observed and noted for euthanasia (weight loss, labored breathing and limb paralysis). Similar to the mouse CAR experiments, 1-week hours after CAR T cell injection bone marrow, spleen and peripheral lymph nodes can be isolated to measure cytokine production and activation markers described above for each ITAM mutant CAR T cell group. Finally, survival studies can be performed with CD19+tumors from patient isolates provided by The Huntsman Cancer Institute at the University of Utah. Additionally, ITAM mutant BCMA CAR T cells can be tested within the NRG mouse model to compare tumor control of the CD19+ and the multiple myeloma BCMA tumors (MM.1S BCMA+ cell line and patient tumor samples).

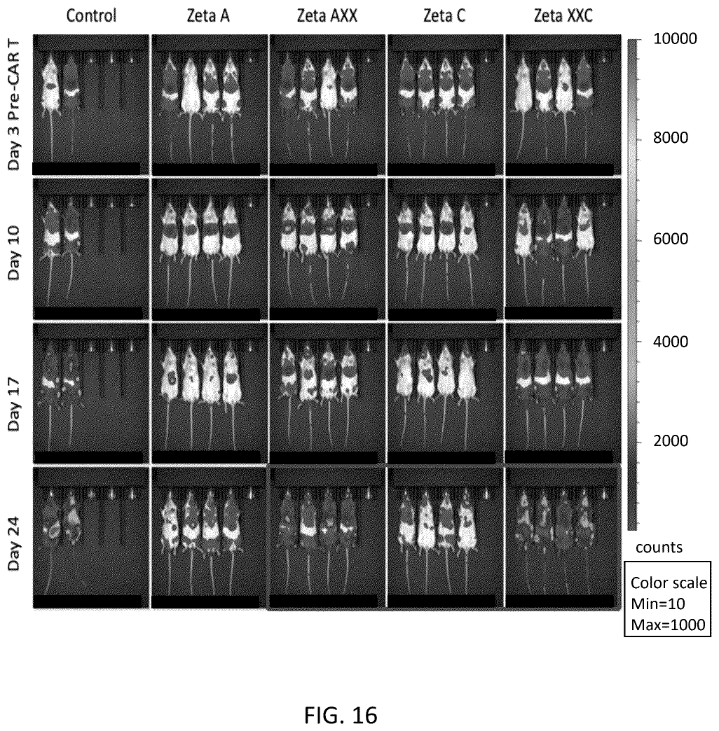

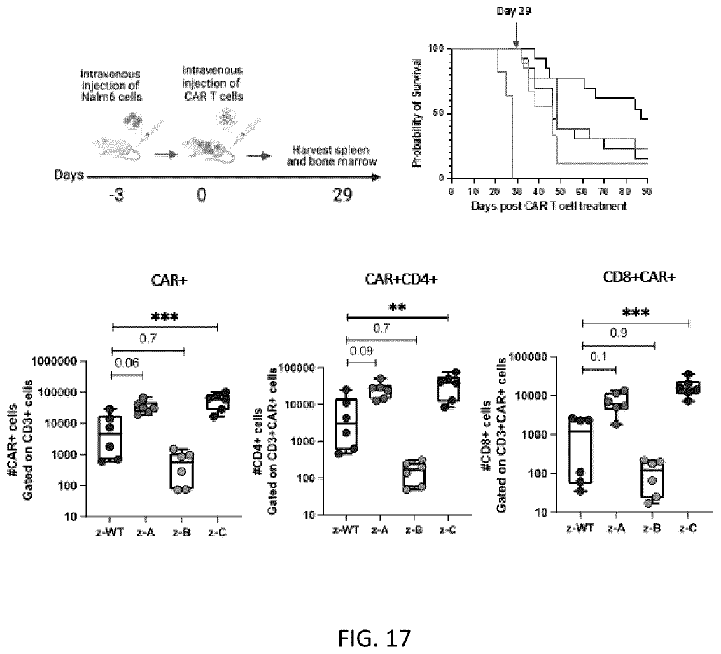

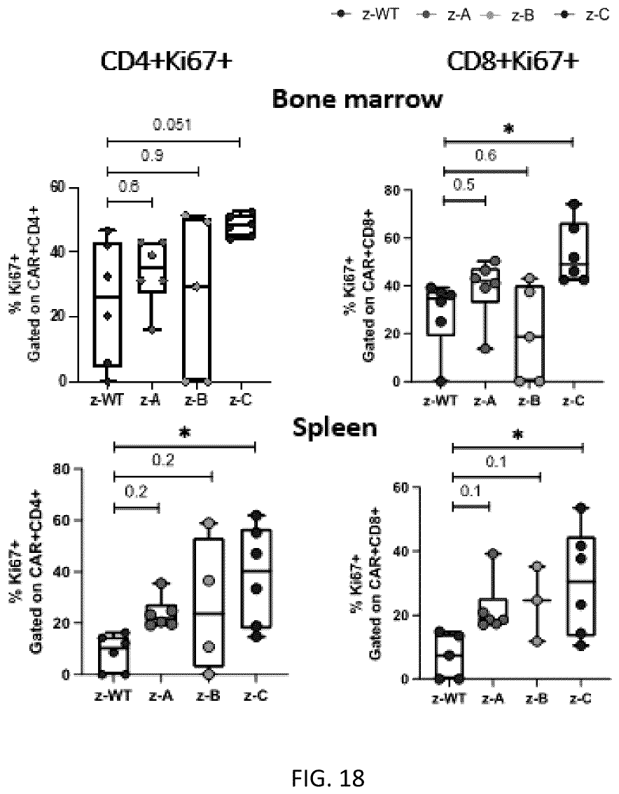

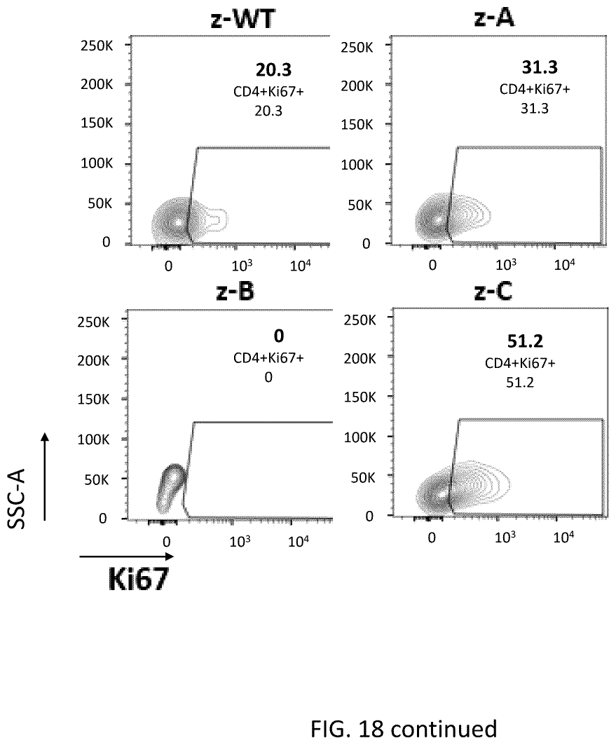

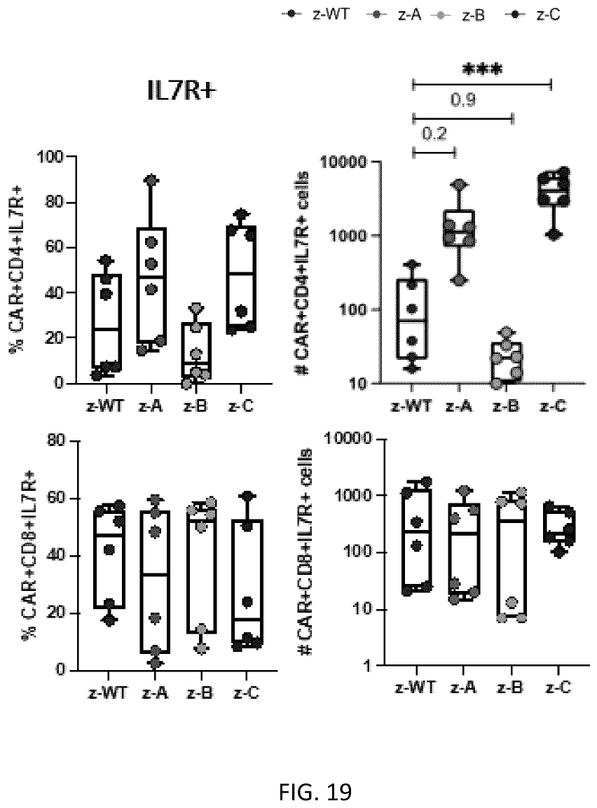

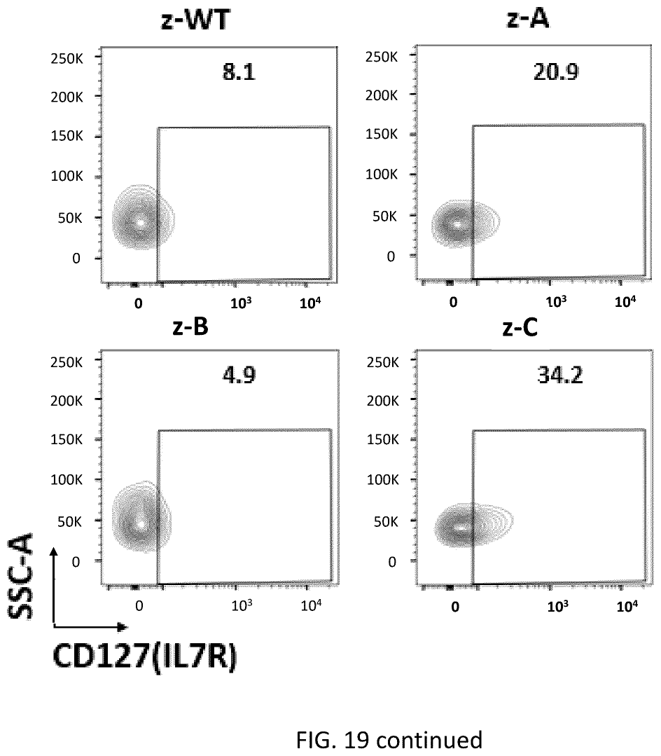

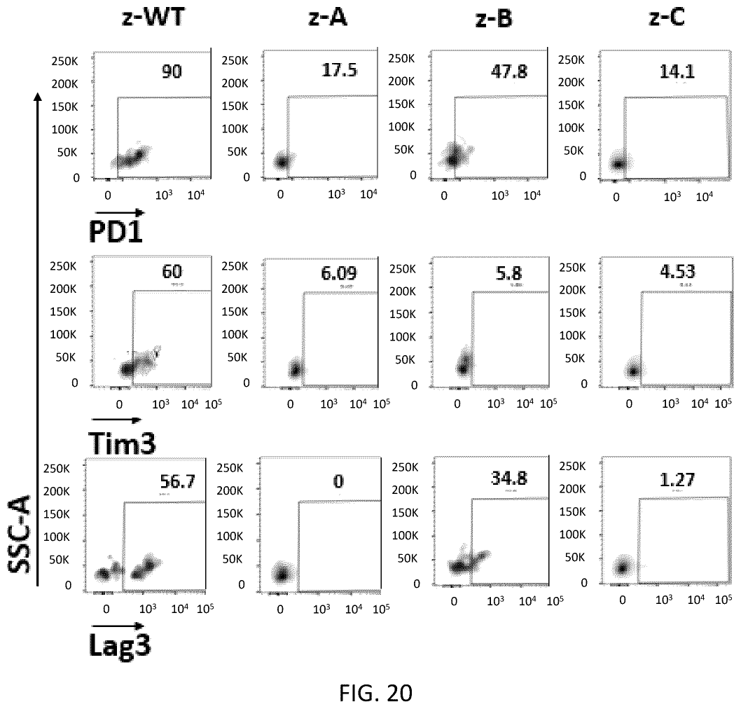

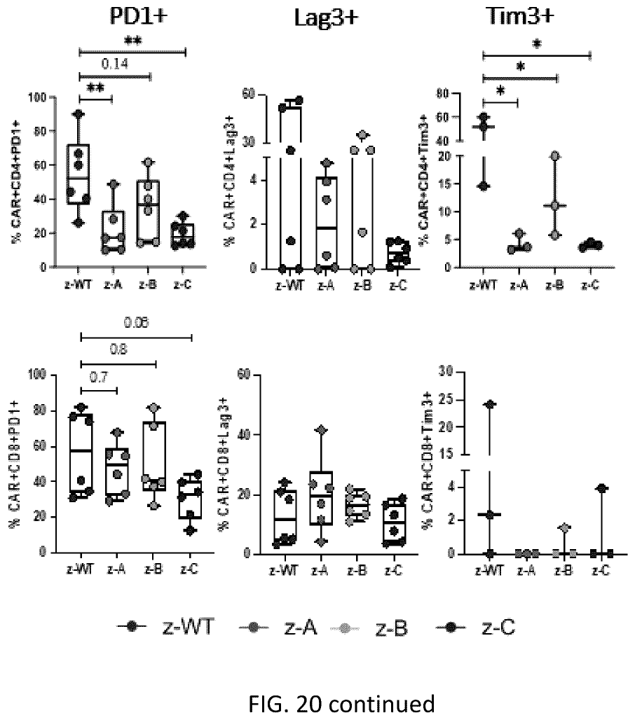

Previously published work demonstrated that limiting the number of functional ITAMS from 3 to 1 had a beneficial impact on CD19 tumor clearance and survival, specifically the z-AXX and z-XXC single ITAM mutants worked the best compared to the WT 3-ITAM construct. Because the z-CCC and z-AAA ITAM mutants displayed different force and bond lifetime measurements compared to the z-AXX and z-XXC constructs, efficacy of tumor clearance was compared. Out to Day 24, it can be demonstrated that the z-CCC construct provide better tumor control compared to the z-XXC and z-AXX (FIG. 16, red box). This early data indicates that both the number and specific sequence of ITAMs may determine a CAR T cells fate.

b. Compare CAR Memory T Cell Development of Human and Mouse CD19 Specific Zeta Chain Mutant CAR T Cells.

To determine CAR T cell persistence and memory fate, C57BL/6 (mouse) and NSG (human) mice will be injected with primary T cells expressing WT, each unique A, B or C ITAMs or No CD3ζ CAR constructs. At days 7, 14, 28, 40, 60, 75 and 80, mice will be sacrificed. The spleens, draining lymph nodes, peripheral blood and bone marrows can be harvested. Lymphocytes isolated can be analyzed by flow cytometry, including memory-cell like markers CD44, CD62L, CD45RA, apoptosis marker Annexin V+ and exhaustion markers PD-1, Tim3, Lag3 by flow cytometry. The transcriptional regulation of genes that control the T cell effector differentiation and memory formation is influenced by the strength of T cell activation and signaling. Gene expression levels of key regulators can be assessed that determine T cell function and fate by bulk RNA seq analysis at early (Day 10) and late (Day 35) time-points. The genes encoding survival and memory-associated regulators can be upregulated in the z-CCC CAR T cells while those for T cell differentiation, effector function and exhaustion can be more highly expressed in z-AAA mutant and z-BBB mutant CARs compared to the WT zeta CAR T cells.

c. Measure Tumor Clearance and Re-Challenge of CD19 and BCMA Positive Tumor Cells within the Mouse and Human CAR T Cells.

Tumor challenge can be performed with Eμ-ALL01 cells in RAG−/− and WT B6 as well as Nalm6 and MM.1S (BCMA+ cell line) with NRG mice at weekly intervals for 3 consecutive challenges and then measure CAR T cell expression of memory-cell like markers CD44, CD62L, exhaustion markers PD-1, Tim3, Lag3 and cytokine production by flow cytometry at day 7 (4 days after first CD19 tumor injection), 14 (first re-challenge), 21 (second re-challenge), 28 (third re-challenge) and then measure persistence and long term memory at days 60 and 75.

### B. Example 2: The Impact of CD3ζ ITAM Multiplicity and Sequence on CAR T-Cell Survival and Function

### 1. Introduction

CARs are artificial receptors that have been designed to recognize user defined antigens in an MHC independent manner and activate downstream signaling pathways that lead to T-cell activation in response to antigen recognition. CAR T-cells have revolutionized the field of cancer immunotherapy with the successful clinical application in the treatment of several refractory B-malignancies like leukemias, lymphomas and multiple myeloma. Current FDA approved CARs consist of an extra-cellular antigen specific antibody derived single-chain variable fragment (scFv), a transmembrane domain, a co-stimulatory domain (CD28 or 41BB) and typically the CD3ζ chain. The CD3ζ chain is phosphorylated at specific conserved tyrosine residues located in signaling motifs called ITAMs to initiate CAR signaling. Despite the early clinical successes, there are several challenges to overcome and further mechanistic studies are required to increase efficacy and the range of cancer treatment.

One of the major challenges facing CAR T-cell therapy is a lack of long-term persistence of CAR T-cells in patients. This is especially true for CD28 based CARs which have been shown to elicit a robust acute anti-tumor response because of stronger signaling strength and faster kinetics. However, similar to the studies with the conventional TCR impact on T-cell function, stronger CAR signaling leads to poor persistence because of more activation induced cell death, early T-cell exhaustion and terminal differentiation. Hence, recent strategies have been focused on the manipulation of the intra-cellular signaling domains of CARs to attenuate signaling strength and improve persistence.

One strategy is to reduce the number of ITAMs in the CD3ζ chain where CAR signaling is initiated. This is based on prior observations with the conventional TCR, where reducing functional ITAM numbers is associated with attenuation of signaling strength and subsequent impact on T cell function. A recent study showed that the single ITAM CAR 1928ζ 1XX had better persistence and tumor killing efficacy with more memory-like CAR T-cells. However, reduction in ITAM number might come at a cost of reduced sensitivity to low antigen density tumors and allow tumor antigen escape. Prior studies with the conventional TCR have also suggested that despite the quantitative significance of ITAM number, there might be qualitative/kinetic differences in the individual ITAM signaling properties based on the amino acid differences surrounding the conserved tyrosine residues. One such study showed that restricting ITAM diversity to single ITAM sequences while maintaining ITAM multiplicity changes TCR signaling strength. Additionally, artificial membrane/liposome based, and computational studies have indicated that there might be inherent differences in the kinetics of tyrosine phosphorylation based on the ITAM sequence. Whether individual CD3 ζ ITAMs A, B, C in CARs differ in their signaling and its effect on CAR T-cell function and persistence is studied herein.

In this study murine 19CD28 (CARs have been generated with restricted CD3 ITAM diversity. Instead of expressing the three different CD3 ζ ITAMs A, B and C as in the conventional CARs (ζABC), the modified CARs express three copies of either A (ζAAA), B (5 BBB) or C (§ CCC). The individual CD3ζ ITAMs can exhibit non-redundant signaling properties in the diversity restricted CARs which may allow for optimization of CAR signaling and improve CAR T-cell persistence and function. The individual CD3 ζ ITAMs confer differential signaling strength to the CARs. The weaker signaling CAR T-cells also have a weaker activation profile. Additionally, the weaker signaling ζCCC are less prone to exhaustion and have better survival under conditions of chronic stimulation. Overall, this study sheds light on the individual signaling tendencies of CD3ζ ITAMs and provides further opportunities for the manipulation of CAR design to reduce CAR T-cell exhaustion and enhance persistence.

### 2. Results

i. Construction of ITAM Restricted Murine Anti-CD19 CARs

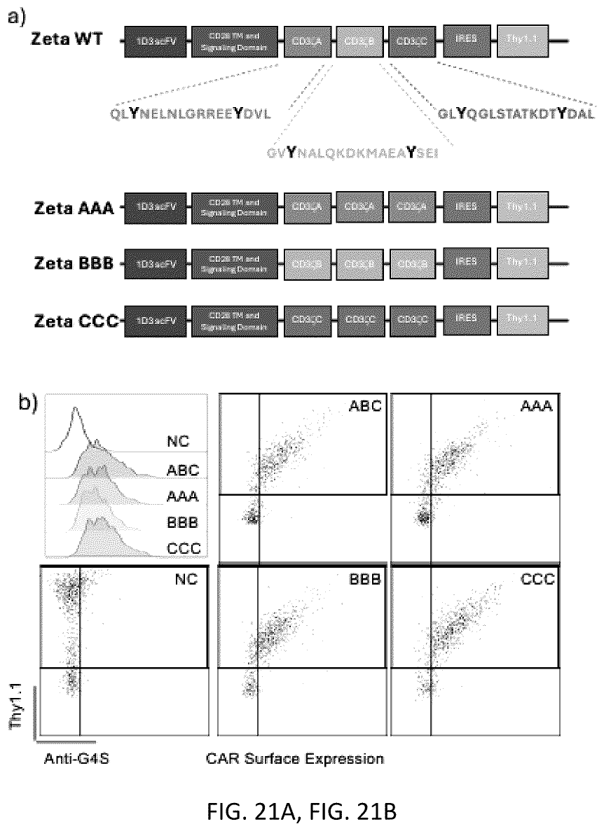

ITAM restricted murine anti-CD19 CD28 CARs were generated that express three copies of a single CD3 ζ ITAM instead of the three different CD3 ζ ITAMs as in the conventional CAR design. The CAR backbone was previously published and obtained from Addgene (Plasmid #107226). It consists of the 1D3 clone anti-CD19 binding scFv domain, a CD28 transmembrane and signaling domain, followed by the CD3 ζ ITAMs (FIG. 21A). The newly designed CARs are designated as ζAAA, ζBBB and ζCCC depending on which CD3ζ ITAM sequence is used. To compare the signaling properties of the ITAM restricted CARs, it was verified that the CAR surface expression levels were similar. Each construct was tested for cell surface expression by retrovirally transducing primary mouse T-cells and staining with an antibody specific for the linker sequence in the CAR construct. Using the anti-G4S antibody, it was observed that the CARs are expressed on the cell surface at similar levels, making signaling studies with these CARs possible (FIG. 21B).

ii. ITAM Restricted CARs Differ in their Signaling Properties

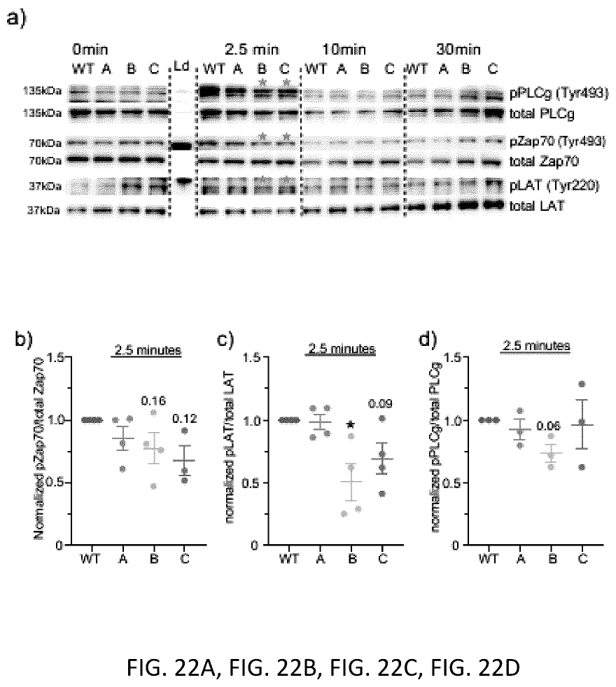

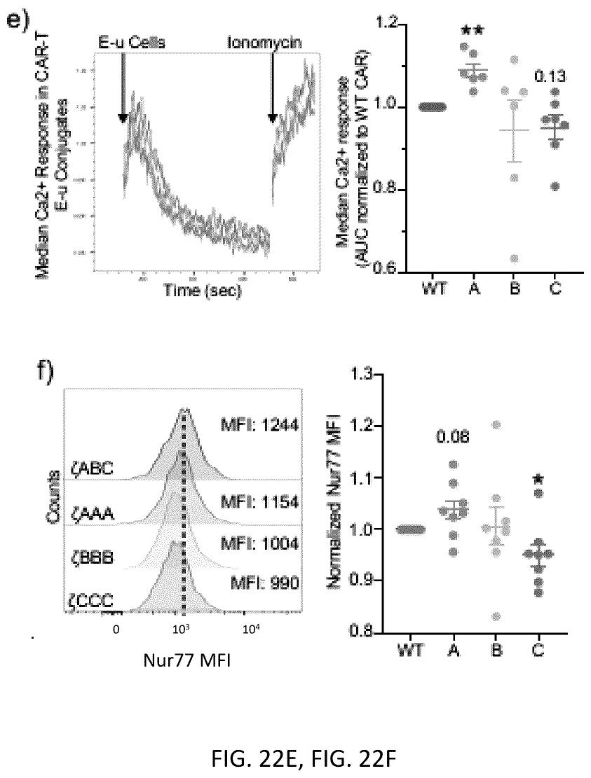

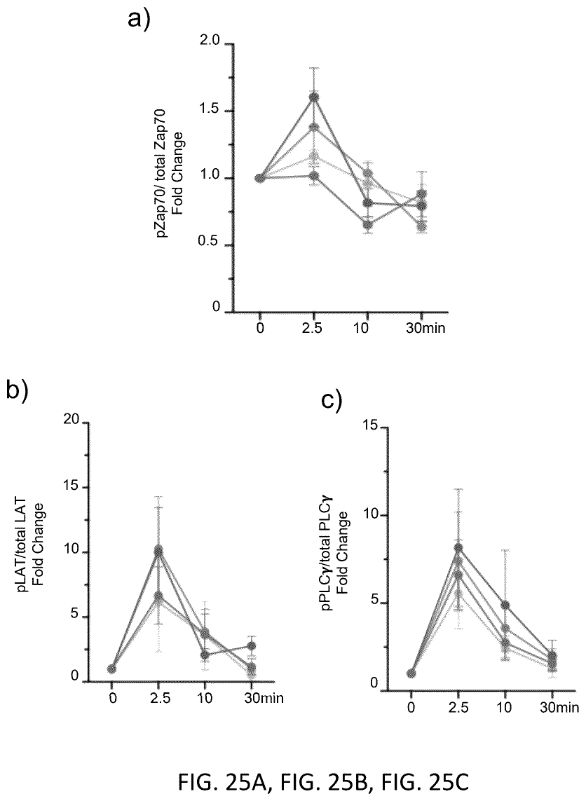

To compare the signaling properties of the individual CD3 ζ ITAMs, the CAR T-cells were stimulated in vitro using CD19 coated dyna-beads for different time-points and quantified the signaling differences using immunoblotting. Phosphorylation of proximal and distal protein targets that have been previously shown to be activated downstream of TCR signaling were measured. The signaling kinetics are similar to that of the TCR with proximal signaling peaking at the 2.5 min timepoint, followed by peaking of distal signaling at the 10 min timepoint (FIG. 22A, FIG. 25A-C). However, at the peak proximal signaling timepoint (2.5 min), it was observed that the proximal signaling molecule LAT was less phosphorylated in (BBB, while pZap70 for & BBB and ζCCC CARs were also slightly decreased in comparison to the ABC CAR, suggesting weaker activation (FIG. 22B-D). To complement this observation, the Ca2+ signaling response was tested in each of the CAR-T cell constructs, which is initiated downstream of PLCγ activation. Ca2+ sensitive Indol-AM labelled CAR-T cells were stimulated with CD19+E-μALL cells and Ca2+ response kinetics was measured using flow cytometry. ζAAA CARs had a significantly greater median Ca2+ response compared to ζABC CARs, while ζCCC trended towards a lower response compared to ζABC CARs (FIG. 22E). To assess the impact of ITAM restricted CARs on downstream transcriptional activity, the expression of Ca2+ responsive transcription factor Nur77 was measured. In concordance with the Ca2+ response, ζCCC CARs were observed to have a significantly lower expression of Nur77 while ζAAA was slightly higher compared to the wild type ITAM CAR (FIG. 22F). Together, these data indicate each ITAM sequence allows for differential CD19-CAR intracellular signaling, therefore it was next wanted to test whether the alteration in signal transduction translated into altered functional outcomes.

iii. ITAM Restricted CARs are Functionally Unique In Vitro

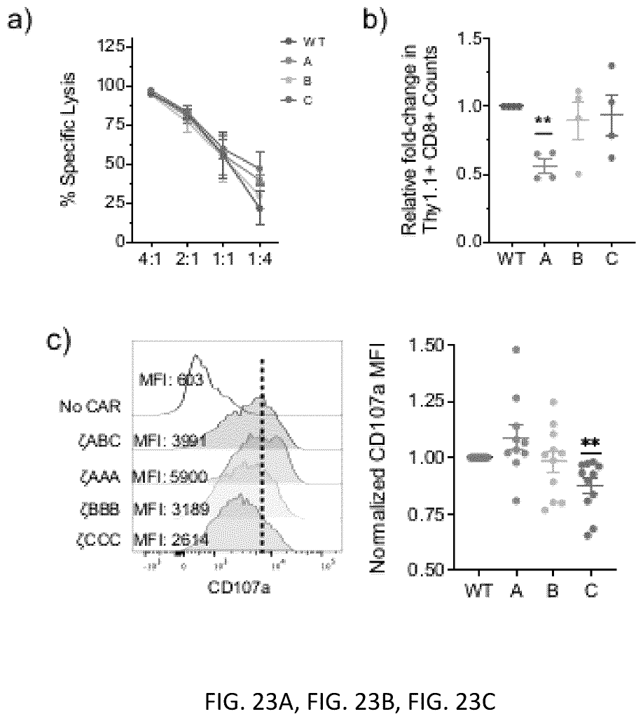

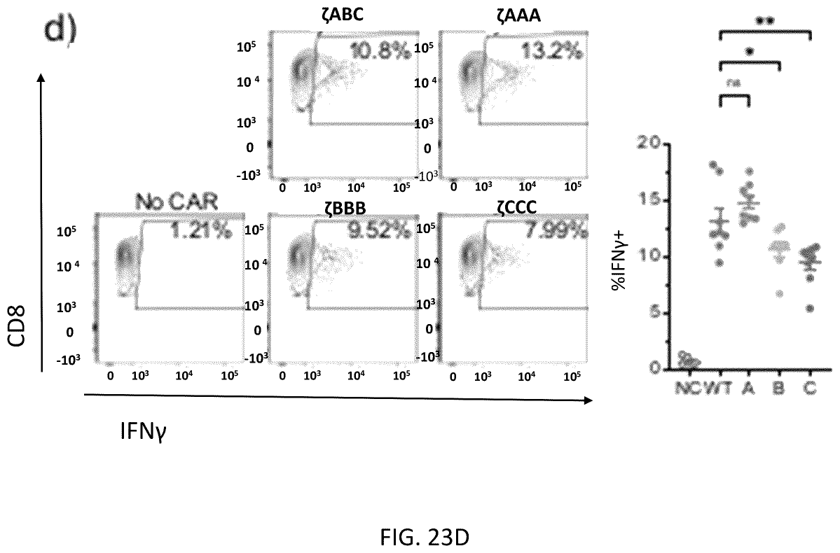

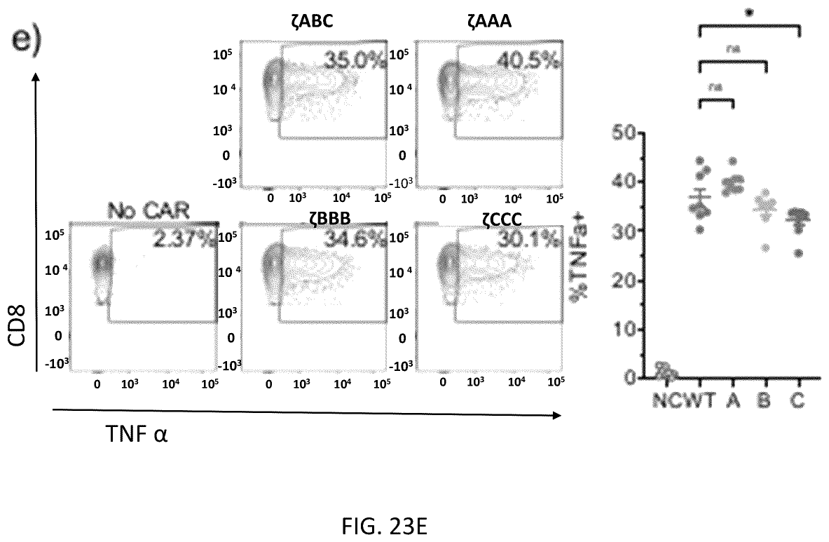

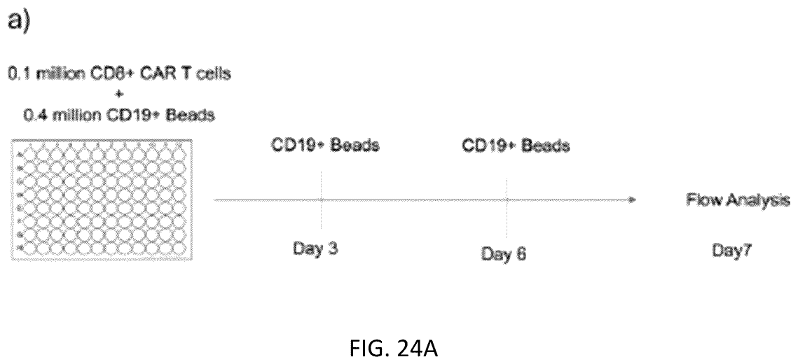

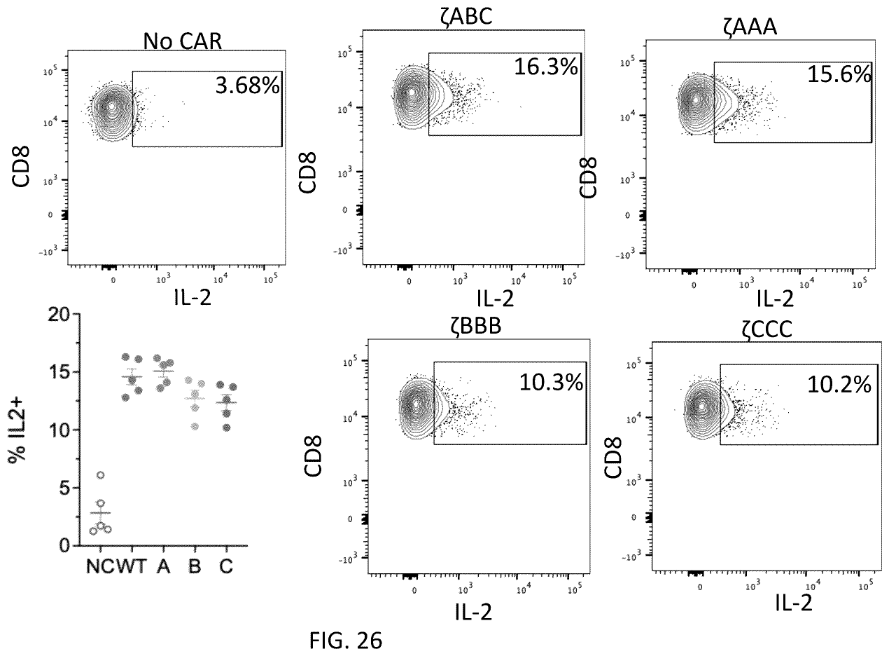

In vitro functional assays were performed to assess the effect of the observed signaling differences on CAR-T function. A dye-release cytotoxicity assay was performed where the CAR-T cells were co-cultured with dye-labelled CD19+E-μtumor cells at different E: T ratios for 16 hours. All of the CARs were similarly effective in their specific cytotoxicity (FIG. 23A). Interestingly, when the viable CD8+CAR T-cell counts were quantified, it was observed ζAAA CARs had reduced cell numbers compared to the ζABC CARs which indicates increased exhaustion or AICD (FIG. 23B). A CD107a degranulation assay was performed to assess lytic effector function. Each ITAM restrict CAR T cell was co-cultured with the CD19+E-μcells in a 1:1 E:T ratio for 6 hours in the presence of PE-conjugated CD107a antibody. Here it was observed ζAAA and ζBBB CARs were similar to ζABC in terms of degranulation (FIG. 23 C). However, ζCCC CARs showed less staining of surface CD107a, suggesting they are less likely to degranulate upon activation (FIG. 23C). Next, an in vitro cytokine expression assay was performed to measure effector cytokine expression of IFNγ, TNFα and IL2. CAR-T cells were co-cultured with CD19+E-μcells for 6 hours in the presence of Brefeldin A and monensin and expression of IFNγ, TNFα and IL2 was measured by intracellular staining. Similar to CD107a expression, ζCCC CARs expressed less IFNγ and TNFα (FIG. 23D-E). However, ζBBB showed a selective decrease in IFNγ expression while maintaining similar levels of TNFα and IL2 expression compared to ζABC (FIG. 23D-E, FIG. 26). Overall, these data indicate that CCC CAR T cells have a weaker activation profile upon encountering tumor antigen that may lead to less effector function but better survival over time.

### iv. ζBBB CAR T-Cells are More Prone to Exhaustion Under Conditions of Chronic Stimulation

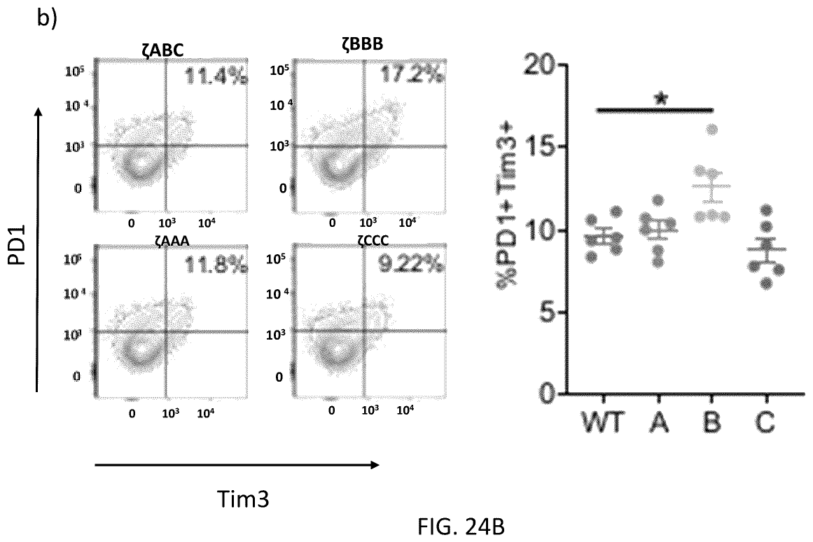

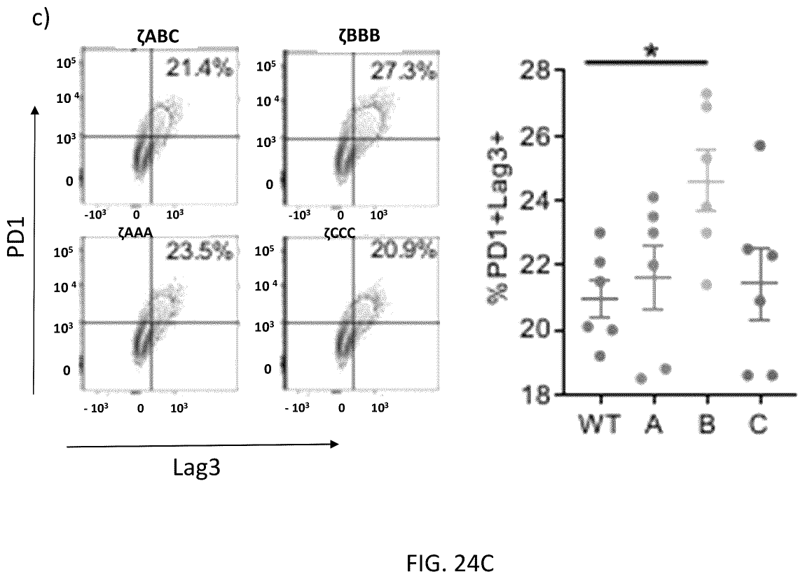

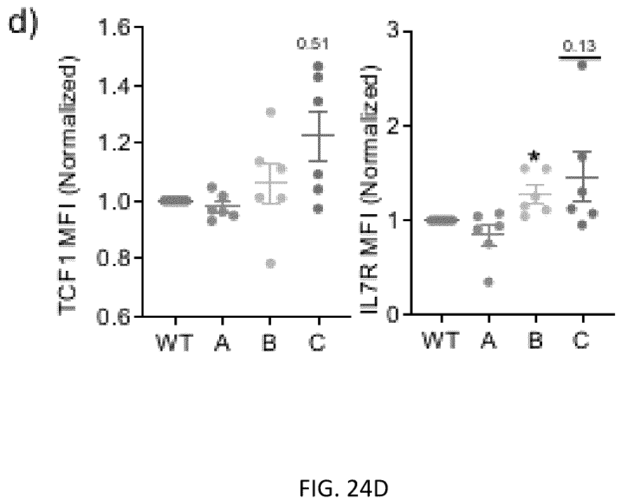

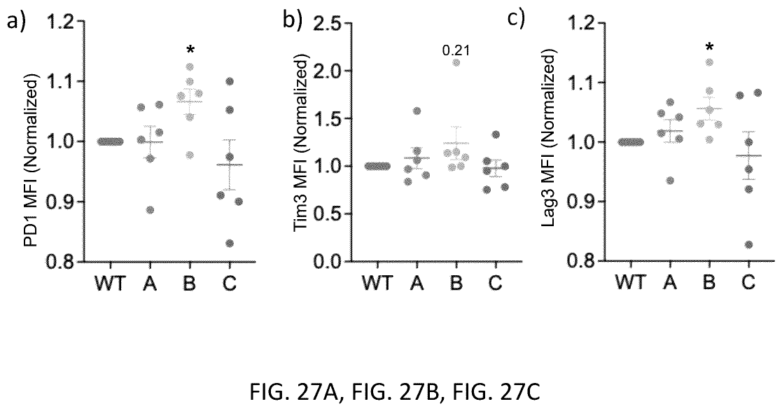

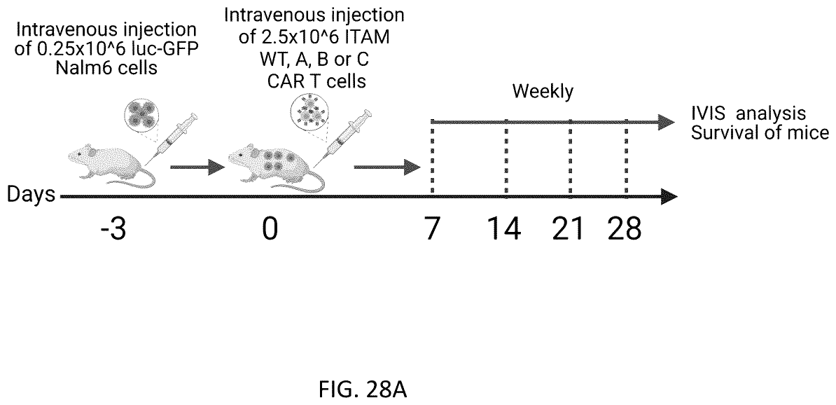

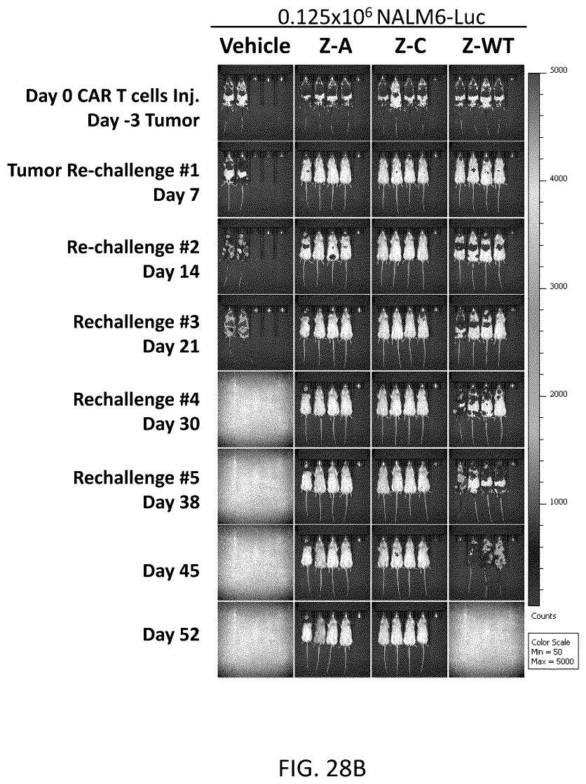

Since previous studies with the conventional T cell have suggested that weaker activation favors less exhaustion, the ITAM restricted CARs were tested for signs of exhaustion in an in vitro exhaustion assay. The CAR T cells were subjected to chronic stimulation conditions by culturing them in the presence of CD19+dyna-beads for 7 days in the presence of IL-2 and analyzed them for expression of exhaustion related inhibitory receptors (FIG. 24A). After repeated stimulation, ζCCC CARs had slightly lower co-expression of inhibitory receptors PD1 and Tim3, whereas & BBB had significantly higher expression compared to ζABC (FIG. 24B-C). Interestingly, ζCCC also exhibited higher expression of TCF1 and IL7R, both of which are associated with less differentiated more memory-like cells (FIG. 27A-C). Together, these data indicate ITAM sequence can lead to differential signal transduction and functional outcomes.

### 3. Discussion

In this study, the signaling properties of the individual CD3 (ITAMs were tested in the context of CD28-based CARs to better understand the impact of ITAM sequence manipulation to improve CAR function persistence through reduction of terminal differentiation, exhaustion and activation induced cell death. In this study, the signaling properties of the individual CD3 ζITAMs were tested in the context of CD28-based CARs to better understand the impact of ITAM sequence manipulation to improve CAR function persistence through reduction of terminal differentiation, exhaustion and activation induced cell death.

It was observed that the ITAM restricted CARs had unique signaling profiles upon stimulation with CD19+beads or tumor cells. The results indicate that the & BBB and ζCCC CARs have weaker phosphorylation/activation of ITAM proximal signaling molecules like Zap70, LAT and PLCγ while ζAAA CARs have comparable levels of activation to that of the & ABC CAR. However, when Ca2+ signaling response was measured, a pathway activated downstream of PLCγ signaling, ζAAA CARs were observed to have higher levels of Ca2+signaling while ζCCC CARs have a trending lower Ca2+ response. Importantly, this effect was reflected at the downstream gene expression level of Nur77, which is a transcription factor whose transcription is regulated by Ca2+ dependent NFAT1-MEF2 co-transcriptional activity. To this end, it was observed that ζCCC CARs had a lower Nur77 expression while ζAAA CARs had a trending higher expression in comparison to ζABC CARs, after CD19+tumor cell stimulation. These observations indicated that ζAAA CARs have an overall stronger signaling strength, while ζCCC CARs have weaker signaling strength. Although differential phosphorylation of downstream signaling targets were shown, further investigation is required to get a deeper understanding of how the amino acid sequences surrounding the conserved tyrosine residues lead to the observed differences. The observed differences are a consequence of preferential binding and differential phosphorylation/dephosphorylation kinetics of the conserved tyrosine residues by the Src family tyrosine kinases Lck/Fyn and the tyrosine phosphatase CD45. This would lead to different rates of Zap70 recruitment and activation of downstream pathways. This would lead to different rates of Zap70 recruitment and activation of downstream pathways. In this regard, the results are in accordance with a previous study, where phosphorylation levels of CD3 ζ ITAM A were observed to be higher than ITAM B and ITAM C by mass spectrometry in Jurkat T-cells activated using anti-CD3 stimulation. Using an on-membrane FRET system, this can be considered a consequence of less efficient dephosphorylation of ITAM A by CD45. There is also the possibility of Zap70 binding to the different SH2 domain binding sites at different rates. This is supported by early studies using synthetic phospho-peptide binding methods, which suggested that Zap70 has a higher binding affinity for doubly phosphorylated ITAM A than ITAMs B and C, with some ambiguity in the hierarchy between ITAMs B and C. It will be of importance to investigate the signalosome of the CAR in future studies to better elucidate the relative protein interactions and binding kinetics of the zeta chain ITAMs.

The unique signaling profiles led to differential T cell functional responses with each ITAM construct. ζCCC CARs had a lower activation profile overall, while ζAAA CARs had a stronger activation profile in terms of the rate of degranulation and expression of effector cytokines IFNγ and TNFα. Although no difference in specific cytotoxicity was detected, less AAA CARs was observed compared to other CARs after tumor killing. This indicates that ζAAA CARs are more prone to activation induced cell death as a result of higher signaling. This would be similar to previous studies indicating conventional TCRs with weaker signaling strength favors less T-cell exhaustion, the in vitro exhaustion assays showed that ζCCC CAR-T cells had lower expression of inhibitory receptors that are associated with exhausted T-cells compared to ζBBB CAR-T cells which may be due to a decrease in Nur77 signaling pathway.

Overall, the results indicate modifying ITAM sequence can regulate CAR signaling strength without having to reduce ITAM multiplicity. Based on the findings it is proposed that ζCCC CARs can perform better in vivo as they have a weaker activation profile and are less prone to exhaustion.

### 4. Materials and Methods

i. Design and Generation of CAR Constructs

MSGV-1D3-285 All ITAMs intact was a gift from James Kochenderfer & Steven Rosenberg (Addgene plasmid #107226; http://n2t.net/addgene: 107226; RRID: Addgene_107226).

ii. Production of Retroviral Supernatant

Platinum-E (Plat-E) cells obtained from ATCC were used as transient retroviral producers. In brief, 1 million Plat-E cells were plated in 10% complete DMEM. The following day, Plat-E cells were transfected with 6 μg vector plasmid using TransIT-LTI (Mirus) transfection reagent in the presence of 25 μM chloroquine. The media was replaced 6 hours later and collected at 48- and 72-hour timepoints for transduction.

iii. T-Cell Isolation, Transduction and Expansion

On Day 0, T-cells were isolated from spleen and lymph nodes (inguinal, axillary and cervical) of 8-12-week-old donor B6 mice. Single-cell suspensions from the organs were subjected to RBC lysis followed by staining with anti-CD8 and/or anti-CD4 biotinylated antibodies. Miltenyi Streptavidin magnetic beads were used to magnetically enrich CD8+ and/or CD4+ T-cells. T-cells were activated in 24-well plates coated with 10 μg/ml anti-CD3 and 2 μg/ml anti-CD28 at a density of ˜0.5 million cells per well. T-cells were stimulated for 24 hours before transduction.

On Day 1, activated T-cells were transduced using retroviral supernatant obtained from the Plat-E cells. Briefly, the cellular debris was removed from the Plat-E supernatant by centrifuging at 300 g for 5 minutes. Polybrene (6 ug/ml) and hIL-2 (50U/ml) were added to the supernatants and cells were spin-transduced at 1300 g for 1 hour at 37° C. After transduction, T-cells were allowed to rest in the viral supernatant for 1 hour in the 37° C. CO2 incubator before replacing media with 10% complete RPMI containing 50U/ml hIL-2. The process was repeated 24 hours later.

After 48-72 hours of activation, on Day 3 the T-cells were removed from the anti-CD3 and anti-CD28 coated plates and transferred to T-cell expansion medium containing 10% complete RPMI supplemented with 10 ng/ml hIL-7 and 10 ng/ml hIL-15. T-cells were maintained at density of ˜0.5 million cells/ml. On Day 5, CAR expressing T-cells were magnetically enriched using anti-Thy1.1 biotinylated antibodies and streptavidin magnetic beads. For in vitro experiments, Day 7-Day 10 cells were used.

iv. Tumor cell line and culture conditions

CD19+E-μALL tumor cell line was obtained from Dr. Marco L. Davila's lab. Since E-μcells cannot expand on their own, they are grown as a co-culture with NIH/3T3 cells. 3T3 cells were cultured in 5% DMEM with ciprofloxacin. One day prior to plating the E-μcells, 3T3 cells were X-ray irradiated at 30Gy and plated at 1 million cells per 10 cm plate. Next day, E-μcells are added and expanded in a 50/50 mix of 10% complete RPMI and 10% complete IMDM.

v. Preparation of CD19+Magnetic Dyna-Beads

M-450 tosylactivated dyna-beads (Invitrogen) were washed and resuspended in 0.1M sodium phosphate buffer (pH 7.8) at 4×108 beads/ml and incubated overnight with 150 μg of recombinant murine CD19 (R&D Systems) with gentle rotation. The beads were then washed 3 times in wash buffer (1× PBS with 0.1% BSA and 2 mM EDTA pH 7.4) at 4° C. for 5 min each with constant mixing. After washing, the beads were stored at 4° C. in storage buffer (1× PBS with 0.02% sodium azide, 0.1% BSA and 2 mM EDTA). Before use, beads were washed 3 times in complete RPMI at 4° C. with constant mixing.

vi. Immunoblotting and Quantification

CAR T-cells were counted and rested in 1×PBS with calcium and magnesium for 30 min at room temperature. Two million CD8+CAR T-cells were stimulated with 6 million CD19+dynabeads by spinning down for Imin and incubating at 37 C for the mentioned timepoints. Signaling was halted by transferring cells to ice and immediately lysing with 1% NP-40 in deionized water containing Halt Protease/Phosphatase inhibitor cocktail. Lysis was performed for 30 min at 4° C. with constant mixing. After spinning down debris, equal volumes of protein lysate were mixed with Laemmli's buffer (Bio-Rad) with 2-mercaptoethanol and denatured at 95° C. for 5 minutes. Samples were then run in pre-cast gels (Invitrogen, NuPage Bis-Tris midi protein gels, 4-12%, 1.0 mm) in MOPS SDS running buffer (Invitrogen). Immunoblotting was performed using Bio-rad's Trans-blot Turbo semi-dry transfer machine onto a PVDF membrane. The membrane was blocked for 1 hour at room temperature using 3% BSA in tris buffered saline with 0.1% Tween20 (0.1% TBS-T). Primary antibody staining was performed overnight at 4° C. in 3% BSA 0.1% TBS-T. Secondary antibody staining was performed at room temperature for 1 hour in 3% BSA 0.1% TBS-T. In between and after staining, the membrane was washed with 0.1% TBS-T 3× for 5 min each. Bands were detected using chemiluminescence. Densitometric analysis was performed using Image Lab 6.1. Protein phosphorylation was quantified as a ratio of phospho band intensity and total protein band intensity. Ration was then expressed as fold change relative to unstimulated cells.

vii. Ca2+ Signaling Assay and Quantification

CD8+CAR T-cells were labelled with the Ca2+ sensitive ratiometric Indo-1AM dye for detection of Ca2+ signaling. Briefly, the CAR-T cells were washed in 1×PBS and incubated with 5 μM Indo-1 AM dye in PBS for 30 min in 37 C incubator. After incubation, the cells were washed twice and resuspended in Ca2+ signaling buffer (Hank's Balanced Salt Solution Cat #55037C with calcium, magnesium and no phenol red, plus 10% FCS and 25 mM HEPES). CAR-T cells were then stained for Thy1.1 for 10 minutes at room temperature. After washing and prior to flow cytometric analysis, the CAR T-cells were stored on ice in 5 ml flow tubes at 1 million cells/500 ul. The E-μcells were labelled with 5 μM Cell Proliferation Dye cFluor™ 670 (cBioscience™) in 1×PBS like the CAR-T cells and stored on ice at 2 million cells/500 ul. Before running each sample on the flow cytometer, the cells were warmed in a 37° C. water-bath for 15 minutes.

Before adding the E-μcells, the CAR-T cells were run on flow cytometer for 1 min to collect baseline Ca2+ bound/Ca2+ free ratio. Flow rate is maintained constant at ˜1000 events/see across samples. At 1 min, the tubes were taken off with recording still ongoing, and 2 million E-u cells were added, mixed at allowed to form THE conjugates by centrifugation at 300 g for 25sec. At the 2 min timepoint, the tube was put back into run mode and data was recorded for 10 minutes to capture Ca2+ response. After 10 minutes, the tube was taken off the cytometer and ionomycin (˜1 ug/ml) was added to check maximum Ca2+ response.

For measurement of Ca2+ response, the experiment gated on Thy 1.1+cf647+conjugates and created a kinetics curve for median of Ca2+ bound/Ca2+ free ratio derived parameter. Gates were set for the peak response (120 sec to 500 sec) and area under curve was calculated and plotted relative to the ζABC CAR.

viii. Dye Release Specific Cytotoxicity Assay

E-μcells were labelled with 5 μM cFluor™ 670 dye in 1×PBS. Thy1.1+CAR T-cells were co-cultured with 50,000 dye labelled E-μcells at the mentioned E: T ratios in a 96-well flat-bottomed plate for ˜15 hours at 37° C. Dye+E-μcell numbers were quantified using flow cytometry and % specific lysis was calculated as % Specific Lysis=((Control cf647+Counts-CAR cf647+Counts))/(Control ef647+Counts)×100

ix. In Vitro Cytokine Stimulation Assay

A total of 100,000 Thy1.1+CAR T-cells were co-cultured with 200,000 E-μcells in 10% complete RPMI at 37° C. for 6 hours in 96 well round-bottomed plates. Brefeldin A (5 μg/ml) and Monensin (2 μM) were added at the beginning of incubation to prevent secretion of cytokines. Cytokine expression was analyzed by intra-cellular staining and flow cytometry.

x. Degranulation Assay

A total of 100,000 Thy1.1+CAR T-cells were co-cultured with 200,000 E-μcells in 10% RPMI at 37° C. in 96-well flat-bottom plates. Phycoerythrin (PE) labelled anti-CD107a (LAMP-1) antibody was added to each well at 1 ug/ml final concentration at the beginning of the incubation to label CD107a exposed on the cell membrane as a result of T cell degranulation.

xi. In Vitro Exhaustion Assay

A total of 100,000 Thy1.1+CD8+CAR T-cells (Day 7 post-activation) were incubated with 400,000 CD19 coated dyna-beads on Day 0 in flat-bottomed 96-well plates in 10% complete RPMI containing 50U/ml IL-2. CAR-T cells were re-stimulated with 400,000 CD19 coated beads at Day 3 and Day 6. Media was replaced every 1.5 days with fresh RPMI containing 50U/ml IL-2. On Day 7, beads were removed, and cells were analyzed by flow cytometry.

xii. Flow Cytometry

Single-cell suspensions were stained in buffer containing PBS, 3% v/v FCS and 0.05% w/v sodium azide. Surface staining was performed on ice for 20 minutes after Fc receptor blocking with FcBlock (Biolegend) for 10 minutes. Live/Dead staining was performed using Zombie Red (Biolegend) in 1×PBS for 30 min on ice. For intra-cellular staining, cells were fixed with FoxP3 Fix/Perm reagent (eBioscience) and then stained under permeabilizing conditions overnight at 4° C. in Permeabilization Buffer (eBioscience). Flow cytometry data were acquired on a BD LSRFortessa (Becton Dickinson) flow cytometer and analyzed using the FlowJo 10 software (FlowJo LLC).

### C. Example 3

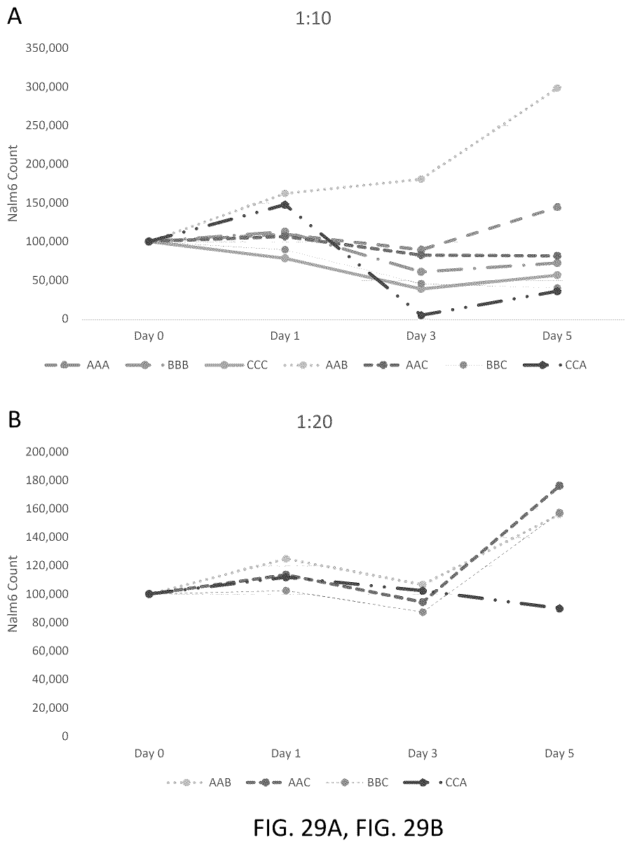

FIG. 29 shows that CD19 specific CAR ITAM combinations AAC, CCA and BBC kill Nalm6 B cell tumors in vitro. Importantly, CCA kills better than AAB, AAC, BBC and very similar to CCC which is the best CAR construct in vivo thus far. This data shows that the 27 unique combinations of the ITAMs can be pursued and is distinct from the AAA, BBB, CCC of the original constructs.

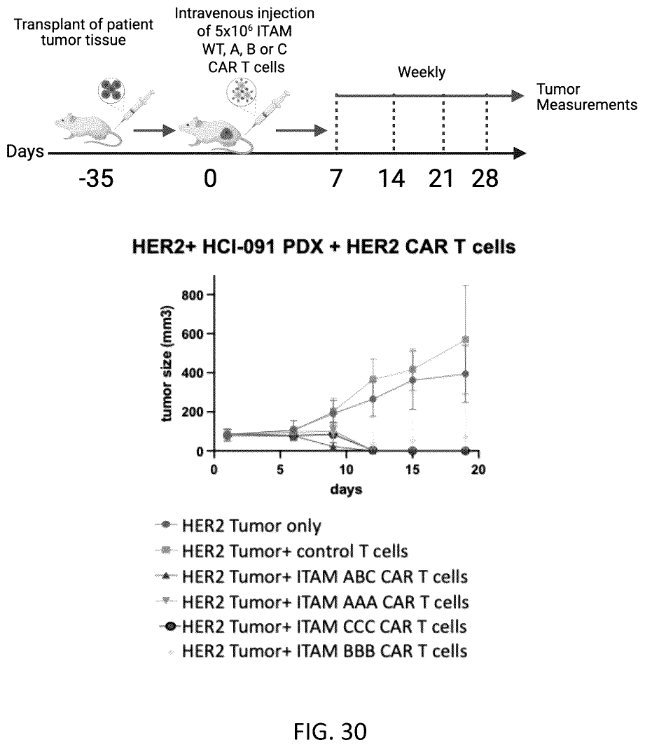

FIG. 30 shows a schematic diagram of a proof of principle that the HER2 specific ITAM mutant CAR T cells can clear a patient derived xenograft (PDX) of a HER2+solid tumor tissue. PDX was implanted and allowed to grow to between 50-100 mm³. Five million human CAR T cells of each ITAM type were injected and tumor sizes were measured two times a week. WT-ABC, AAA and CCC cleared the human PDX tumors, while the control T cell group (not CAR expression) could not.

Those skilled in the art will recognize, or be able to ascertain using no more than routine experimentation, many equivalents to the specific embodiments of the method and compositions described herein. Such equivalents are intended to be encompassed by the following claims.

1. A human chimeric antigen receptor (CAR) polypeptide comprising an antigen binding domain, a transmembrane domain, and an intracellular signaling domain, wherein the intracellular signaling domain comprises a variant CD3 zeta (CD3ζ).

2. The human CAR polypeptide of claim 1, wherein the variant CD3ζ comprises three Immunoreceptor tyrosine activation motifs (ITAMs).

3. The human CAR polypeptide of claim 2, wherein the variant CD3ζ comprises two or more of ITAMs CD3ζa, CD3ζb, or CD3 ζc.

4. The human CAR polypeptide of claim 2, wherein the variant CD3ζ does not comprise one or more of ITAMs CD3ζa, CD3ζb, and CD3ζc.

5. The human CAR polypeptide of claim 2, wherein the variant CD3ζ comprises one or two, but not all three of ITAMs CD3ζa, CD3ζb, and CD3 ζc.

6. The human CAR polypeptide of claim 2, wherein the variant CD3ζ comprises three CD3ζc ITAMs,
wherein the variant CD3ζcomprises three CD3ζb ITAMs, or
wherein the variant CD3ζcomprises three CD3ζa ITAMs.

7. (canceled)

8. (canceled)

9. The human CAR polypeptide of claim 1, wherein the variant CD3ζcomprises two CD3ζa ITAMs and one CD3ζb ITAM,
wherein the variant CD34 comprises two CD3ζa ITAMs and one CD3ζc ITAM,
wherein the variant CD3ζcomprises two CD3ζb ITAMs and one CD3ζa ITAM,
wherein the variant CD3ζ comprises two CD3ζb ITAMs and one CD3ζc ITAM,
wherein the variant CD3 ζcomprises two CD3ζc ITAMs and one CD3ζa ITAM, or
wherein the variant CD3 comprises two CD3ζc ITAMs and one CD3ζb ITAM.

10.-14. (canceled)

15. The human CAR polypeptide of claim 3, wherein the one or more CD3ζa ITAM comprises the amino acid sequence of QLYNELNLGRREEYDVL, or a variant thereof,
wherein the one or more CD3ζb ITAM comprises the amino acid sequence of GLYNELQKDKMAEAYSEI, or a variant thereof, and/or
wherein the one or more CD3ζc ITAM comprises the amino acid sequence of GLYQGLSTATKDTYDAL, or a variant thereof.

16.-17. (canceled)

18. The CAR polypeptide of claim 1, wherein the antigen binding domain is a CD19 binding domain.

19. The CAR polypeptide of claim 18, wherein the antigen binding domain is a single-chain variable fragment (scFv) of an antibody that specifically binds CD19, wherein the CDR1 sequence of the V_{H} domain comprises the amino acid sequence SEQ ID NO: 5; the CDR2 sequence of the V_{H} domain comprises the amino acid sequence SEQ ID NO: 6; the CDR3 sequence of the V_{H} domain comprises the amino acid sequence SEQ ID NO:7; the CDR1 sequence of the V_{L} comprises the amino acid sequence SEQ ID NO: 8; the CDR2 sequence of the V_{L} domain comprises the amino acid sequence SEQ ID NO:9; and the CDR3 sequence of the V_{L} domain comprises the amino acid sequence SEQ ID NO:10;

20. The human CAR polypeptide of claim 18, wherein the CD19 binding domain comprises the sequence of
(SEQ ID NO: 4)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLELKRGGGGSGGGGSGGGGSGGGGSEVQLQQSGPGLVAPSQSLSVTC
TVSGVSLPDYGVSWIRQPPRKGLEWLGVIWGSETTYYNSALKSRLTIIKD
NSKSQVFLKMNSLQTDDTAIYYCAKHYYYGGSYAMDY.

21. The human CAR polypeptide of claim 1, wherein the antigen binding domain is an antibody fragment or an antigen-binding fragment that specifically binds to a target antigen.

22. (canceled)

23. The human CAR polypeptide of claim 1, wherein the intracellular signaling domain comprises a co-stimulatory signaling region.

24. The human CAR polypeptide of claim 23, wherein the co-stimulatory signaling region comprises the cytoplasmic domain of a costimulatory molecule selected from the group consisting of 4-1BB, CD28, CD27, OX40, CD30, CD40, PD-1, ICOS, lymphocyte function-associated antigen-1 (LFA-1), CD2, CD7, LIGHT, NKG2C, B7-H3, CD137, DAP10a ligand that specifically binds with CD83, and any combination thereof.

25. The human CAR polypeptide of claim 1, wherein the intracellular signaling domain comprises the variant CD3 signaling domain and a co-stimulatory signaling region, wherein the co-stimulatory signaling region comprises the cytoplasmic domain of CD28 or 4-1BB.

26. The human CAR polypeptide of claim 1, wherein the transmembrane domain comprises a transmembrane domain of a protein chosen from the alpha, beta, or zeta chain of T-cell receptor, CD28, OX40, H2-Kb, CD3 epsilon, CD45, CD4, CD5, CD7, CD8, CD9, CD16, CD22, CD33, CD37, CD64, CD80, CD86, CD134, CD137, CD154, or immunoglobulin Fc domain.

27.-33. (canceled)

34. A nucleic acid sequence capable of encoding the human CAR polypeptide of claim 1.

35.-38. (canceled)

39. A cell comprising the human CAR polypeptide of claim 1.

40.-44. (canceled)

45. A method of treating cancer comprising administering a therapeutically effective amount of a composition comprising a T cell genetically modified to express the human CAR polypeptide of claim 1 to a subject having cancer.

46. (canceled)

47. A method of reducing tumor growth in a subject having cancer comprising administering a therapeutically effective amount of a T cell genetically modified to express the human CAR polypeptide of claim 1 to the subject.

48.-51. (canceled)


SEQUENCE LISTING (21 sequences)

SEQ ID NO: 1 - protein, 17 aa, synthetic construct
QLYNELNLGRREEYDVL

SEQ ID NO: 2 - protein, 18 aa, synthetic construct
GLYNELQKDKMAEAYSEI

SEQ ID NO: 3 - protein, 17 aa, synthetic construct
GLYQGLSTATKDTYDAL

SEQ ID NO: 4 - protein, 237 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDY

SEQ ID NO: 5 - protein, 10 aa, synthetic construct
GVSLPDYGVS

SEQ ID NO: 6 - protein, 16 aa, synthetic construct
VIWGSETTYYNSALKS

SEQ ID NO: 7 - protein, 13 aa, synthetic construct
KHYYYGGSYAMDY

SEQ ID NO: 8 - protein, 11 aa, synthetic construct
RASQDISKYLN

SEQ ID NO: 9 - protein, 7 aa, synthetic construct
HTSRLHS

SEQ ID NO: 10 - protein, 9 aa, synthetic construct
QQGNTLPYT

SEQ ID NO: 11 - DNA, 30 bp, synthetic construct
  source 1..30: mol_type = other DNA
ggggtctcattacccgactatggtgtaagc

SEQ ID NO: 12 - DNA, 48 bp, synthetic construct
  source 1..48: mol_type = other DNA
gtaatatggggtagtgaaaccacatactataattcagctctcaaatcc

SEQ ID NO: 13 - DNA, 39 bp, synthetic construct
  source 1..39: mol_type = other DNA
aaacattattactacggtggtagctatgctatggactac

SEQ ID NO: 14 - DNA, 33 bp, synthetic construct
  source 1..33: mol_type = other DNA
agggcaagtcaggacattagtaaatatttaaat

SEQ ID NO: 15 - DNA, 21 bp, synthetic construct
  source 1..21: mol_type = other DNA
catacatcaagattacactca

SEQ ID NO: 16 - DNA, 27 bp, synthetic construct
  source 1..27: mol_type = other DNA
caacagggtaatacgcttccgtacacg

SEQ ID NO: 17 - DNA, 855 bp, synthetic construct
  source 1..855: mol_type = other DNA
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcg

SEQ ID NO: 18 - DNA, 51 bp, synthetic construct
  source 1..51: mol_type = other DNA
caactttataatgaacttaacctcggtcgtcgagaagaatacgatgttcta

SEQ ID NO: 19 - DNA, 54 bp, synthetic construct
  source 1..54: mol_type = other DNA
ggcctgtacaatgaactgcagaaagataagatggcggaggcctacagtgagatt

SEQ ID NO: 20 - DNA, 51 bp, synthetic construct
  source 1..51: mol_type = other DNA
ggcctttaccagggtctcagtacagccaccaaggacacctacgacgccctt

SEQ ID NO: 21 - protein, 285 aa, synthetic construct
MEFGLSWLFLVAILKGVQCSRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSA